(19) United States
(12) Patent Application Publication
(10) Pub. No.: US 2008/0004329 A1
(43) Pub. Date: Jan. 3, 2008

(54) Pharmaceutical compositions of ropinirole and methods of use thereof

(75) Inventors: Gene Jamieson, Boulder Creek, CA (US); Dario Norberto Carrara, Oberwill (CH); Arnaud Grenier, Steinbrunn le haut (FR)

Correspondence Address: JAZZ PHARMACEUTICALS, 3180 PORTER DRIVE, PALO ALTO, CA, 94304, US

(73) Assignee: Jazz Pharmaceuticals, Palo Alto, CA (US)

(21) Appl. No.: 11/823,028

(22) Filed: Jun. 26, 2007

Related U.S. Application Data
(60) Provisional application No. 60/817,259, filed on Jun. 29, 2006.

(51) Int. Cl. A61K 31/40; A61P 43/00

(52) U.S. Cl. 514/418

(57) ABSTRACT

The present invention comprises compositions for pharmaceutical drug delivery of an indolone (e.g., ropinirole), or a pharmaceutically acceptable salt thereof. The composition may, for example, be a gel suitable for transdermal application. The compositions of the present invention typically comprise a hydroalcoholic vehicle, one or more antioxidant, and one or more buffering agent, wherein the pH of the gel is usually between about pH 7 and about pH 9. The compositions may include further components, for example, the hydroalcoholic vehicle may further comprise additional solvent(s), antioxidant(s), cosolvent(s), penetration enhancer(s), buffering agent(s), and/or gelling agent(s). The compositions may be used for the treatment of a variety of neurological disorders.

This application claims the benefit of priority, under 35 U.S.C. 119(e), to U.S. Provisional Application Ser. No. 60/817,259, filed Jun. 29, 2006 and is incorporated herein by reference.

### TECHNICAL FIELD

The present invention relates to formulations, including compositions and dosage forms, of indolone derivatives and their salts, for example, ropinirole, and pharmaceutically acceptable salts thereof. Described herein are formulations that are useful and efficacious for transdermal delivery, as well as methods of use and methods of manufacturing for such formulations.

### BACKGROUND OF THE INVENTION

Transdermal delivery is a noninvasive, convenient method that can provide a straightforward dosage regimen, relatively slow release of the drug into a patient's system, and control over blood concentrations of the drug. In contrast to oral administration, transdermal delivery typically does not produce variable rates of metabolism and absorption, and it causes no gastrointestinal side effects. In addition, transdermal delivery is ideal for patients who cannot swallow medication and for drugs with significant metabolism in the liver.

Transdermal delivery also poses inherent challenges, in part because of the nature of skin. Skin is essentially a thick membrane that protects the body by acting as a barrier. Consequently, the movement of drugs or any external agent through the skin is a complex process. The structure of skin includes the relatively thin epidermis, or outer layer, and a thicker inner layer called the dermis. For a drug to penetrate unbroken skin, it must first move into and through the stratum corneum, which is the outer layer of the epidermis. Then the drug must penetrate the viable epidermis, papillary dermis, and capillary walls to enter the blood stream or lymph channels. Each tissue features a different resistance to penetration, but the stratum corneum is the strongest barrier to the absorption of transdermal and topical drugs. The tightly packed cells of the stratum corneum are filled with keratin. The keratinization and density of the cells may be responsible for skin's impermeability to certain drugs.

In recent years, advances in transdermal delivery include the formulation of permeation enhancers (skin penetration enhancing agents). Permeation enhancers often are lipophilic chemicals that readily move into the stratum corneum and enhance the movement of drugs through the skin. Non-chemical modes also have emerged to improve transdermal delivery; these include ultrasound, iontophoresis, and electroporation. But even with these methodologies, only a limited number of drugs can be administered transdermally without problems such as sensitization or irritation occurring.

Transdermal delivery should not be confused with topical treatment. Transdermal drugs are absorbed through skin or mucous membranes to provide effects beyond the application site. In contrast, the goal with a topical drug, e.g., antibiotic ointment, is to administer medication at the site of intended action. Topical medications typically do not cause significant drug concentrations in the patient's blood and/or tissues. Topical formulations are often used to fight infection or inflammation. They also are used as cleansing agents, astringents, absorbents, keratolytics, and emollients. The base of a topical treatment, the component that carries the active ingredient(s), may interact with the active ingredient(s), changing the drug's effectiveness. Thus, the base must be selected with care. The base and/or active ingredient(s) may cause skin irritation or allergic reactions in some patients. Topical formulations may be prepared as creams, ointments, lotions, solutions, or aerosols. Occlusive therapy may be used with topical treatments to improve the drug's absorption and effectiveness. In occlusive therapy, the topical treatment is applied to the skin and covered, for example, with household plastic wrap, bandages, or plastic tape.

The present invention is directed to the transdermal administration of certain indolone derivatives and their salts, for example, ropinirole, and pharmaceutically acceptable salts thereof (see, e.g., U.S. Pat. Nos. 4,452,808, 4,824,860, 4,906,463, 4,912,126, and 5,807,570). Ropinirole is a novel dopamine D₂ agonist indicated for use in treating a number of disorders, including, but not limited to, Parkinson's Disease, Restless Legs Syndrome, Tourette's Syndrome, Chronic Tic Disorder, Essential Tremor and Attention Deficit Hyperactivity Disorder. Ropinirole has a molecular weight of 296.84 and a melting point of approximately 247° C. Ropinirole hydrochloride has a solubility of 133 mg/ml in water at 20° C.

Parkinson's Disease is a progressive disorder of the nervous system that affects neurons in the part of the brain that controls muscle movement. Symptoms include trembling, muscle rigidity, difficulty walking, and problems with balance and coordination. Ropinirole overcomes the limitations of L-Dopa therapy in the treatment of Parkinson's Disease and has been identified as a more specific dopamine D₂ agonist than dopamine agonists such as pergolide and bromocriptine.

Restless Legs Syndrome is a neurological movement condition characterized by uncomfortable sensations in the legs such as itching, tingling, twitching, cramping or burning as well as a compelling urge to move the legs to relieve the discomfort. Symptoms typically intensify when the patient is lying down, making it difficult to sleep.

Tourette's Syndrome is a neurological disorder characterized by tics, involuntary vocalizations and movements such as facial twitches and eye blinks. These compelled movements and vocalizations may occur many times a day or intermittently over the span of a year or more. A related condition, Chronic Tic Disorder, is characterized by rapid, recurrent, uncontrollable movements or by vocal outbursts.

Essential Tremor is another neurological disorder. Tremor is involuntary trembling in part of the body. Essential Tremor is associated with purposeful movement, for example, shaving, writing, and holding a glass to drink. Most often Essential Tremor occurs in the hands and head. It may also affect the larynx, arms, body trunk, and legs of an affected patient. It is believed that Essential Tremor is caused by abnormalities in areas of the brain that control movement. It does not occur as the result of disease (e.g., Parkinson's disease) nor does it usually result in serious complications.

Attention Deficit Hyperactivity Disorder (ADHD) is characterized by hyperactivity, distractibility, forgetfulness, poor impulse control, and mood shifts. ADHD commonly is diagnosed among children.

The formulations of the present invention as described herein below provide a number of advantages for the transdermal delivery of ropinirole and its derivatives. These include, but are not limited to, continuous, steady-state delivery, which can provide sustained blood levels of the agent(s).

### SUMMARY OF THE INVENTION

In one aspect, the present invention relates to compositions (for example, a gel) for pharmaceutical drug delivery. In one embodiment, the composition may be formulated to be suitable for transdermal application. The composition typically comprises a therapeutically effective amount of an indolone, or a pharmaceutically acceptable salt thereof. A preferred indolone is ropinirole, or a pharmaceutically acceptable salt thereof. Further, the composition may be a gel. The gel typically comprises a primary vehicle comprising a mixture of water and at least one short-chain alcohol (i.e., a hydroalcoholic vehicle), one or more antioxidant; and one or more buffering agent. The apparent pH of the gel is usually between about pH 1 and about pH 8.5, and the gel is adapted for application to the surface of skin. The compositions for pharmaceutical delivery may include further components as described herein, for example, the hydroalcoholic vehicle may further comprise additional solvent(s), antioxidant(s), cosolvent(s), penetration enhancer(s), buffering agent(s), and/or gelling agent(s).

Preferred embodiments of the present invention are gel formulations for non-occlusive therapeutic, transdermal applications.

The formulations of the present invention may be provided, for example, in unit dose container(s) or multiple dose containers.

In another aspect, the present invention comprises a composition for pharmaceutical drug delivery. Such compositions may, for example, comprise a therapeutically effective amount of ropinirole, or a pharmaceutically acceptable salt thereof, a hydroalcoholic vehicle, and at least one buffering agent. In such compositions the pH of the composition is between about pH 7 and about pH 8.5. Further, the transdermal flux of the ropinirole, in the hydroalcoholic vehicle, across skin is greater than the transdermal flux of an equal concentration of ropinirole in an aqueous solution of essentially equivalent pH over an essentially equivalent time period, wherein the skin acts as the flux rate controlling membrane.

In yet another aspect the present invention comprises a composition for pharmaceutical drug delivery. Such compositions may, for example, comprise a therapeutically effective amount of ropinirole, or a pharmaceutically acceptable salt thereof, in a hydroalcoholic vehicle. In such compositions the ropinirole has an apparent pKa of about 8.0 or less compared to a theoretical pKa of ropinirole in water of about pKa 9.7.

The above-described compositions for pharmaceutical delivery may include further components as described herein, for example, the hydroalcoholic vehicle may further comprise additional solvent(s), antioxidant(s), cosolvent(s), penetration enhancer(s), buffering agent(s), and/or gelling agent(s).

The compositions of the present invention may be used, for example, for transdermal applications including application to skin and mucosal tissue (for example, intranasally, or as a suppository).

In yet another aspect, the present invention includes dosage forms for pharmaceutical delivery of a drug, for example, ropinirole. In one embodiment, the dosage form is configured to provide steady-state delivery of ropinirole with once-a-day dosing. The steady-state ratio of Cₘₐₓ/Cₘᵢₙ in such dosage forms may be, for example, less than about 1.75 when the subject's plasma level concentration of ropinirole is at steady-state (C_{SS}). In another embodiment of the present invention, the steady-state oscillation of Cₘₐₓ to Cₘᵢₙ in such dosage forms may be, for example, greater than about 8 hours when the subject's plasma level concentration of ropinirole is at steady-state (C_{SS}).

In a further aspect, the present invention includes methods of manufacturing the compositions described herein for pharmaceutical drug delivery.

In another aspect, the present invention includes methods for administering an active agent to a subject in need thereof. For example, the method may comprise providing a composition of the present invention for transdermal, pharmaceutical delivery of ropinirole. Ropinirole, and pharmaceutical salts thereof, can be used for the treatment of a variety of conditions including, but not limited to, movement disorders. Exemplary conditions/disorders include, but are not limited to, neurological disorders, often including, but not limited to, Parkinson's Disease, Restless Legs Syndrome, Tourette's Syndrome, Chronic Tic Disorder, Essential Tremor, and Attention Deficit Hyperactivity Disorder.

These and other embodiments of the present invention will readily occur to those of ordinary skill in the art in view of the disclosure herein.

### BRIEF DESCRIPTION OF THE FIGURES

FIG. 1 shows data for flux results from the permeation analysis using the formulations in described in Example 1.

FIG. 2 presents the mass balance recovery data from the permeation analysis shown in FIG. 1.

FIG. 3 shows data for the absolute kinetic delivery profile of ropinirole delivery over the 24 hour permeation period using the formulations described in Example 2.

FIG. 4A presents a profile of ropinirole delivery compared to the theoretical ionization profile of ropinirole. FIG. 4B presents an experimental ionization profile of ropinirole.

FIG. 5 shows data for the absolute kinetic delivery profile of ropinirole delivery over the 24 hour permeation period using the formulations described in Example 4.

FIG. 6 shows data for the absolute kinetic delivery profile of ropinirole delivery over the 24 hour permeation period using the formulations described in Example 5.

FIG. 7 shows the results of ropinirole instant flux over the 24 hour permeation period using the formulations described in Example 5.

FIG. 8 shows the data for ropinirole bioavailability over a 24 hour permeation period for the formulations described in Example 6. The plotted data shows the relative kinetic profile for ropinirole permeation.

FIG. 9 presents the data for ropinirole transdermal delivery relative to the apparent ionization profile of ropinirole.

FIG. 10 presents data for the absolute kinetic delivery profile over a 24 hour permeation period for the formulations described in Example 7.

FIG. 11 presents data for ropinirole flux over a 24 hour permeation period for the formulations described in Example 7.

FIG. 12 presents modeling results showing predicted plasma concentration over one week period for three-time per day oral administration of ropinirole for 5 consecutive days.

FIG. 13 presents modeling results showing predicted plasma concentration over one week period for a once-a-day ropinirole transdermal administration for 5 consecutive days.

FIG. 14 shows the actual profile of plasma ropinirole following treatment with ropinirole during Day 1.

FIG. 15 shows the actual profile of plasma ropinirole following treatment with ropinirole for five days.

### DETAILED DESCRIPTION OF THE INVENTION

All patents, publications, and patent applications cited in this specification are herein incorporated by reference as if each individual patent, publication, or patent application was specifically and individually indicated to be incorporated by reference in its entirety for all purposes.

1.0.0 Definitions

It is to be understood that the terminology used herein is for the purpose of describing particular embodiments only, and is not intended to be limiting. As used in this specification, description of specific embodiments of the present invention, and any appended claims, the singular forms “a,” “an” and “the” include plural referents unless the context clearly dictates otherwise. Thus, for example, reference to “a cosolvent” includes two or more cosolvents, mixtures of cosolvents, and the like, reference to “a compound” includes one or more compounds, mixtures of compounds, and the like.

Unless defined otherwise, all technical and scientific terms used herein have the same meaning as commonly understood by one of ordinary skill in the art to which the invention pertains. Although other methods and materials similar, or equivalent, to those described herein can be used in the practice of the present invention, the preferred materials and methods are described herein.

In describing and claiming the present invention, the following terminology will be used in accordance with the definitions set out below.

The term “dosage form” as used herein refers to a pharmaceutical composition comprising an active agent, such as ropinirole, and optionally containing inactive ingredients, e.g., pharmaceutically acceptable excipients such as suspending agents, surfactants, disintegrants, binders, diluents, lubricants, stabilizers, antioxidants, osmotic agents, colorants, plasticizers, coatings and the like, that may be used to manufacture and deliver active pharmaceutical agents.

The term “gel” as used herein refers to a semi-solid dosage form that contains a gelling agent in, for example, an aqueous, alcoholic, or hydroalcoholic vehicle and the gelling agent imparts a three-dimensional cross-linked matrix (“gellified”) to the vehicle. The term “semi-solid” as used herein refers to a heterogeneous system in which one solid phase is dispersed in a second liquid phase.

The pH measurements for formulations and compositions described herein, wherein the formulations or compositions do not comprise a predominantly aqueous environment, are more aptly described as “apparent pH” values as the pH values are not determined in a predominantly aqueous environment. In such cases, the influence of, for example, organic solvents on the pH measurement may result in a shift of pH relative to a true aqueous environment.

The term “carrier” or “vehicle” as used herein refers to carrier materials (other than the pharmaceutically active ingredient) suitable for transdermal administration of a pharmaceutically active ingredient. A vehicle may comprise, for example, solvents, cosolvents, permeation enhancers, pH buffering agents, antioxidants, gelling agents, additives, or the like, wherein components of the vehicle are nontoxic and do not interact with other components of the total composition in a deleterious manner.

The phrase “non-occlusive, transdermal drug delivery” as used herein refers to transdermal delivery methods or systems that do not occlude the skin or mucosal surface from contact with the atmosphere by structural means, for example, by use of a patch device, a fixed application chamber or reservoir, a backing layer (for example, a structural component of a device that provides a device with flexibility, drape, or occlusivity), a tape or bandage, or the like that remains on the skin or mucosal surface for a prolonged period of time. Non-occlusive, transdermal drug delivery includes delivery of a drug to skin or mucosal surface using a topical medium, for example, creams, ointments, sprays, solutions, lotions, gels, and foams. Typically, non-occlusive, transdermal drug delivery involves application of the drug (in a topical medium) to skin or mucosal surface, wherein the skin or mucosal surface to which the drug is applied is left open to the atmosphere.

The term “transdermal” delivery, as used herein refers to both transdermal (or “percutaneous”) and transmucosal administration, that is, delivery by passage of a drug through a skin or mucosal tissue surface and ultimately into the bloodstream.

The phrase “therapeutically effective amount” as used herein refers to a nontoxic but sufficient amount of a drug, agent, or compound to provide a desired therapeutic effect, for example, one or more doses of ropinirole that will be effective in relieving symptoms of a neurological disorder, often including, but not limited to, a movement disorder (e.g., Parkinson's Disease, Restless Legs Syndrome, Tourette's Syndrome, Chronic Tic Disorder, Essential Tremor, and Attention Deficit Hyperactivity Disorder).

The term “ropinirole” as used herein refers to ropinirole free base, pharmaceutically acceptable salts thereof, as well as mixtures of free base and salt forms. One example of a pharmaceutically acceptable salt of ropinirole is the hydrochloride salt of 4-[2-(dipropylamino)-ethyl]-1,3-dihydro-2H-indol-2-one monohydrochloride, which has an empirical formula of C₁₆H₂₄N₂O.HCl. The molecular weight of ropinirole HCl is approximately 296.84 (260.38 as the free base). The structure of ropinirole HCl is as follows:

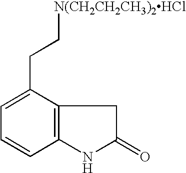

The phrase “ropinirole free base equivalent” as used herein typically refers to the actual amount of the ropinirole molecule in a formulation, that is, independent of the amount of the associated salt forming compound that is present in a ropinirole salt. The phrase ropinirole free base equivalent may be used to provide ease of comparison between formulations made using ropinirole free base or any of a number of ropinirole salts to show the amount of active ingredient (e.g., ropinirole) that is present in the formulation. For example, free base ropinirole has a molecular weight of approximately 260.38. Ropinirole HCl has a molecular weight of approximately 296.84 of which approximately 36.46 of the molecular weight is attributed to HCl. The molecular weight ratio of ropinirole HCl to free base ropinirole is 1.14. Accordingly, when ropinirole HCl is present in a formulation at 3.42 weight percent this corresponds to a ropinirole free base equivalent of 3 weight percent (3.42/1.14=3.00).

The term “indolone derivatives and their salts” as used herein refers to compounds, and pharmaceutically acceptable salts thereof, generally having the following structure:

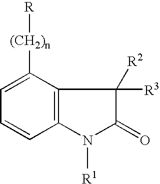

wherein, R is amino, lower alkylamino, di-lower alkylamino, allylamino, diallylamino, N-lower alkyl-N-allylamino, benzylamino, dibenzylamino, phenethylamino, diphenethylamino, 4-hydroxyphenethylamino or di-(4-hydroxyphenethylamino), R1, R2 and R3 are each hydrogen or lower alkyl, and n is 1-3.

The phrase “short-chain alcohol” as used herein refers to a C₂-C₄ alcohol, for example, ethanol, propanol, isopropanol, and/or mixtures of thereof.

The phrase “volatile solvent” refers to a solvent that changes readily from solid or liquid to a vapor, and that evaporates readily at normal temperatures and pressures. Examples of volatile solvents include, but are not limited to, ethanol, propanol, isopropanol, and/or mixtures thereof. The term “non-volatile solvent” as used herein refers to a solvent that does not change readily from solid or liquid to a vapor, and that does not evaporate readily at normal temperatures and pressures. Examples of non-volatile solvents include, but are not limited to, propylene glycol, glycerin, liquid polyethylene glycols, polyoxyalkylene glycols, and/or mixtures thereof. Stanislaus, et al., (U.S. Pat. No. 4,704,406) defined “volatile solvent” as a solvent whose vapor pressure is above 35 mm Hg when skin temperature is 32° C., and a “non-volatile” solvent as a solvent whose vapor pressure is below 10 mm Hg at 32° C. skin temperature. Solvents used in the practice of the present invention are typically physiologically compatible and used at non-toxic levels.

The phrase “permeation enhancer” or “penetration enhancer” as used herein refers to an agent that improves the rate of transport of a pharmacologically active agent (e.g., ropinirole) across the skin or mucosal surface. Typically, a penetration enhancer increases the permeability of skin or mucosal tissue to a pharmacologically active agent. Penetration enhancers, for example, increase the rate at which the pharmacologically active agent permeates through skin and enters the bloodstream. Enhanced permeation effected through the use of penetration enhancers can be observed, for example, by measuring the flux of the pharmacologically active agent across animal or human skin as described in the Examples herein below. An “effective” amount of a permeation enhancer as used herein means an amount that will provide a desired increase in skin permeability to provide, for example, the desired depth of penetration of a selected compound, rate of administration of the compound, and amount of compound delivered.

The phrase “stratum corneum” as used herein refers to the outer layer of the skin. The stratum corneum typically comprises layers of terminally differentiated keratinocytes (made primarily of the proteinaceous material keratin) arranged in a brick and mortar fashion wherein the mortar comprises a lipid matrix (containing, for example, cholesterol, ceramides, and long chain fatty acids). The stratum corneum typically creates the rate-limiting barrier for diffusion of the active agent across the skin.

The phrase “intradermal depot” as used herein refers to a reservoir or deposit of a pharmaceutically active compound within or between the layers of the skin (e.g., the epidermis, including the stratum corneum, dermis, and associated subcutaneous fat), whether the pharmaceutically active compound is intracellular (e.g., within keratinocytes) or intercellular.

The term “subject” as used herein refers to any warm-blooded animal, particularly including a member of the class Mammalia such as, without limitation, humans and nonhuman primates such as chimpanzees and other apes and monkey species; farm animals such as cattle, sheep, pigs, goats and horses; domestic mammals such as dogs and cats; laboratory animals including rodents such as mice, rats and guinea pigs, and the like. The term does not denote a particular age or sex.

The term “sustained release ” as used herein refers to predetermined continuous release of a pharmaceutically active agent to provide therapeutically effective amounts of the agent over a prolonged period. In some embodiments of the present invention, the sustained release occurs at least in part from an intradermal depot of a pharmaceutically active compound.

The term “prolonged period” as used herein typically refers to a period of at least about 12 hours, more preferably at least about 18 hours, and more preferably at least about 24 hours.

The term “sustained release dosage form” as used herein refers to a dosage form that provides an active agent, e.g., ropinirole, substantially continuously for several hours, typically for a period of at least about 12 to about 24 hours.

The term “delivery rate” as used herein refers to the quantity of drug delivered, typically to plasma, per unit time, for example, nanograms of drug released per hour (ng/hr) in vivo.

In the context of plasma blood concentration of active agent, the term “C” as used herein refers to the concentration of drug in the plasma of a subject, generally expressed as mass per unit volume, typically nanograms per milliliter (this concentration may be referred to as “plasma drug concentration” or “plasma concentration” herein which is intended to be inclusive of drug concentration measured in any appropriate body fluid or tissue). The plasma drug concentration at any time following drug administration is typically referred to as Cₜᵢₘₑ as in C₁₀ₕ or C₂₀ₕ, etc. The term “Cₘₐₓ” refers to the maximum observed plasma drug concentration following administration of a drug dose, and is typically monitored after administration of a first dose and/or after steady-state delivery of the drug is achieved. The following terms are used herein as follows: “C_{avg}” refers to average observed plasma concentration typically at steady state, C_{avg} at steady state is also referred to herein as “C_{SS}”; “Cₘᵢₙ” refers to minimum observed plasma concentration typically at steady state.

The term “Tₘₐₓ” as used herein refers to the time to maximum plasma concentration and represents the time that elapses between administration of the formulation and a maximum plasma concentration of drug (i.e., a peak in a graph of plasma concentration vs. time, see, for example, FIG. 13). Tₘₐₓ values may be determined during an initial time period (for example, related to administration of a single dose of the drug) or may refer to the time period between administration of a dosage form and the observed maximum plasma concentration during steady state.

The term “steady state” as used herein refers to a pattern of plasma concentration versus time following consecutive administration of a constant dose of active agent at predetermined intervals (for example, once-a-day dosing). During “steady state” the plasma concentration peaks and plasma concentration troughs are substantially the same within each dosing interval.

One of ordinary skill in the art appreciates that plasma drug concentrations obtained in individual subjects will vary due to inter-subject variability in many parameters affecting, for example, drug absorption, distribution, metabolism, and excretion. Accordingly, mean values obtained from groups of subjects are typically used for purposes of comparing plasma drug concentration data and for analyzing relationships between in vitro dosage assays and in vivo plasma drug concentrations.

2.0.0 General Overview of the Invention

Before describing the present invention in detail, it is to be understood that this invention is not limited to particular embodiments described herein, for example, particular solvent(s), antioxidant(s), cosolvent(s), penetration enhancer(s), buffering agent(s), and/or gelling agent(s), and the like, as use of such particulars may be selected in view of the teachings of the present specification by one of ordinary skill in the art. It is also to be understood that the terminology used herein is for the purpose of describing particular embodiments of the invention only, and is not intended to be limiting.

In one aspect, the present invention relates to a gel composition for pharmaceutical drug delivery. The gel may be formulated to be suitable for transdermal application, for example, transcutaneous and/or transmucosal applications. The gel typically comprises a therapeutically effective amount of an indolone, or a pharmaceutically acceptable salt thereof. A preferred indolone is ropinirole, or a pharmaceutically acceptable salt thereof. The gel typically comprises a primary vehicle comprising a mixture of water and at least one short-chain alcohol, one or more antioxidant; and one or more buffering agent, wherein (i) the pH of the gel is between about pH 7 and about pH 8.5, and (ii) the gel is suitable for application to the surface of skin of a subject. In one embodiment, the ropinirole is free base ropinirole. In other embodiments, the ropinirole is a pharmaceutically acceptable salt of ropinirole (e.g., ropinirole HCl). A preferred concentration range of ropinirole is about 0.5 to about 10 weight percent of ropinirole free base equivalents, more preferred is a concentration of about 1 to about 5 weight percent of ropinirole free base equivalents.

The short-chain alcohol in formulations of the present invention may be, for example, ethanol, propanol, isopropanol, and mixtures thereof. A preferred concentration range of the short-chain alcohol, for example, ethanol, is a concentration of about 30 to about 70 weight percent where the water is present at a concentration of about 10 to about 60 weight percent. Water can be added quantum sufficiat (q.s.) so amounts may vary as can be determined by one of ordinary skill in the art in view of the teachings of the present specification. A more preferred concentration range of the short-chain alcohol, for example, ethanol, is about 40 to about 60 weight percent where the water is present at a concentration of about 10 to about 40 weight percent.

The gel formulations of the present invention may further comprise a non-volatile solvent (for example, a glycol or glycerin). In one embodiment the glycol is propylene glycol. A preferred concentration range of the non-volatile solvent(s), for example, propylene glycol, is a concentration of about 10 to about 60 weight percent, more preferred is a concentration of about 15 to about 40 weight percent.

Further, the gel formulations of the present invention may further comprise a gelling agent(s). Exemplary gelling agents include, but are not limited to, modified cellulose (for example, hydroxypropyl cellulose, hydroxyethyl cellulose, and carboxymethyl cellulose), and gums. A preferred concentration range of the gelling agent(s), for example, hydroxypropyl cellulose, is a concentration of between about 0.5 and about 5 weight percent, more preferred is a concentration of between about 1 and about 3 weight percent.

The gel formulations of the present invention may also further comprise a permeation enhancer (penetration enhancer). A preferred concentration range of the penetration enhancer(s), is a concentration of between about 0.1 and about 10 weight percent, more preferred is a concentration of between about 1 and about 7 weight percent. In one embodiment, the penetration enhancer comprises a mixture of diethylene glycol monoethylether and myristyl alcohol in, respectively, a 5:1 ratio weight/weight.

A preferred concentration range of the antioxidant(s) of the gel formulations of the present invention, for example, sodium metabisulfite, is a concentration of about 0.01 to about 5 weight percent; more preferred is a concentration of about 0.1 to about 0.5 weight percent.

A preferred concentration range of the buffering agent(s) of the gel formulations of the present invention, for example, triethanolamine, is a concentration of about 1 to about 10 weight percent, more preferred is a concentration of about 3 to about 5 weight percent. Concentrations of buffering agents may vary, however, as described further herein below.

In one embodiment, a gel formulation of the present invention comprises, a therapeutically effective amount of ropinirole, or a pharmaceutically acceptable salt thereof, of between about 0.5 to about 5 weight percent of ropinirole free base equivalents. The primary vehicle may comprise between about 10 to about 60 weight percent of water, between about 30 to about 70 weight percent ethanol, between about 10 and about 60 weight percent propylene glycol, and between about 0.1 and about 10 weight percent of a 5:1 (weight to weight) mixture of diethylene glycol monoethylether and myristyl alcohol. The primary vehicle may be gellified with between about 0.5 and about 5 weight percent of hydroxypropyl cellulose. The antioxidant comprises between about 0.01 and about 5 weight percent of sodium metabisulfite. Further, the buffering agent comprises triethanolamine between about 1 to about 10 weight percent, wherein the pH of the gel is between about pH 7 and about pH 9, or preferably between about pH 7 and pH 8.5.

Preferred embodiments of the present invention are gel formulations for non-occlusive therapeutic, transdermal applications. In such embodiments, transdermal delivery methods or systems do not occlude the skin or mucosal surface from contact with the atmosphere by structural means, for example, there is no backing layer used to retain the gel formulation in place on skin or mucosal surface.

The formulations of the present invention may be provided in a unit dose container(s). Such containers typically comprise inner and outer surfaces, wherein the formulation of the present invention is contained by the inner surface of the container. In selected embodiments, the container is a packet or a vial, and the inner surface of the container may further comprise a liner. For example, in one embodiment, the container is a flexible, foil packet and the liner is a polyethylene liner. Alternatively, or in addition, the formulations of the present invention may be provided in a multiple dose container(s). Such multiple dose containers typically comprise inner and outer surfaces, wherein the gel for pharmaceutical drug delivery is contained by the inner surface of the container. Multiple dose containers may, for example, dispenses fixed or variable metered doses. Multiple dose containers may, for example, be a stored-energy metered dose pump or a manual metered dose pump.

In another aspect, the present invention comprises a composition for pharmaceutical drug delivery, comprising a therapeutically effective amount of ropinirole, or a pharmaceutically acceptable salt thereof, in a hydroalcoholic vehicle comprising water, a short chain alcohol, and at least one buffering agent. In such compositions the pH of the composition is typically between about pH 7 and about pH 8.5. Further, the transdermal flux (for example, instant flux) of the ropinirole, in the hydroalcoholic vehicle, across skin is greater than the transdermal flux of an equal concentration of ropinirole in an aqueous solution (that is, a solution without the short-chain alcohol solvent or other cosolvent) of essentially equivalent pH over an essentially equivalent time period, wherein the skin is the flux rate controlling membrane. These compositions for pharmaceutical delivery may include further components as described herein, for example, the hydroalcoholic vehicle may further comprise an antioxidant(s). Such compositions may be formulated in a variety of ways including wherein the hydroalcoholic vehicle is gellified. These compositions may be used, for example, for transdermal applications including application to skin and mucosal tissue (for example, intranasally, or as a suppository).

In yet another aspect the present invention comprises a composition for pharmaceutical drug delivery, comprising a therapeutically effective amount of ropinirole, or a pharmaceutically acceptable salt thereof, in a hydroalcoholic vehicle comprising water, and a short chain alcohol. In such compositions the ropinirole has an apparent pKa of about 8.0 or less compared to a theoretical pKa of ropinirole in water of about pKa 9.7. In some embodiments, the ropinirole is a pharmaceutically acceptable salt (for example, ropinirole HCl). These compositions for pharmaceutical delivery may include further components as described herein, for example, the hydroalcoholic vehicle may further comprise an antioxidant(s), a cosolvent(s), a penetration enhancer(s), a buffering agent(s), and/or a gelling agent(s). Such compositions may be formulated in a variety of ways including wherein the hydroalcoholic vehicle is gellified. These compositions may be used, for example, for transdermal applications including application to skin and mucosal tissue (for example, intranasally, or as a suppository).

In a further aspect, the present invention includes methods of manufacturing the compositions described herein for pharmaceutical drug delivery. In one embodiment, the method of manufacturing comprises mixing the components to yield a homogeneous gel, wherein the pH of the gel is between about pH 7 and about pH 8.5 (exemplary components include, but are not limited to the following: a therapeutically effective amount of ropinirole, or a pharmaceutically acceptable salt thereof; a primary vehicle comprising water, at least one short-chain alcohol, and at least one gelling agent; at least one antioxidant; and at least one buffering agent). These methods may include addition of further components as described herein, for example, the hydroalcoholic vehicle may further comprise an antioxidant(s), a cosolvent(s), a penetration enhancer(s), a buffering agent(s), and/or a gelling agent(s). The method provides a gel suitable for pharmaceutical delivery of ropinirole. Further, a method of manufacturing may further include dispensing the pharmaceutical composition into one or more containers (for example, a unit dose container (e.g., a flexible, foil packet, further comprising a liner) or a multiple dose container).

In another aspect, the present invention includes methods for administering an active agent to a human subject in need thereof. For example, the method may comprise providing a composition of the present invention for transdermal, pharmaceutical delivery of ropinirole. Doses of the compositions of the present invention may, for example, be a gel applied to the surface of skin. Further, doses of the compositions of the present invention may be applied in a single or in divided doses. In one embodiment, the composition is applied as one or more daily dose of the gel to a skin surface of the subject in an amount sufficient for the ropinirole to achieve therapeutic concentration in the bloodstream of the subject. The divided doses may be applied at intervals of 6, 8, 12 or 24 hours. Ropinirole, and pharmaceutical salts thereof, can be used for the treatment of a variety of conditions including neurological disorders, for example, movement disorders. Exemplary conditions/disorders include, but are not limited to, Parkinson's Disease, Restless Legs Syndrome, Tourette's Syndrome, Chronic Tic Disorder, Essential Tremor, and Attention Deficit Hyperactivity Disorder. In one embodiment, the composition is a gel that has an amount of ropinirole free base equivalents between about 3 and about 5 weight percent, wherein up to about 1.0 grams of the gel is applied daily to a skin surface area of between about 50 to about 1000 cm². In another embodiment, the composition is a gel that has an amount of ropinirole free base equivalents of about 1.5 weight percent, wherein up to about 1.5 grams of the gel is applied daily to a skin surface area of between about 70 to about 300 cm². In yet another embodiment, the composition is a gel that has an amount of ropinirole free base equivalents of about 3 weight percent, wherein 0.25 grams of gel is applied to a skin surface of between about 50 and 300 cm².

In another aspect, the present invention includes dosage forms for delivery of ropinirole that provide therapeutically effective steady-state plasma ropinirole concentration to a subject. In one embodiment, the steady-state plasma level is achieved by once-a-day dosing. With once-a-day dosing the maximum attained plasma concentration may be achieved more than about 24 hours after administration (that is, after administration of a second consecutive dose). The sustained release provided by this dosage form also provides a reduced ratio of Cₘₐₓ to Cₘᵢₙ relative to oral dosage forms administered more than once a day. The dosage form of the present invention is, in one embodiment, designed to be a once-a-day dosage form that provides continuous treatment of, for example, movement disorders through delivery of therapeutically effective amounts of ropinirole over 24 hours.

Embodiments of the present invention include a dosage form for delivery of ropinirole to a subject comprising, a dose of ropinirole, wherein said dosage form is configured to provide steady-state delivery of ropinirole with once-a-day dosing. The dosage form provides a steady-state ratio of Cₘₐₓ/Cₘᵢₙ that is less than about 1.75, more preferably less than about 1.5, and more preferably less than about 1.3, when the subject's plasma level concentration of ropinirole is at steady-state (C_{SS}). The once-a-day dosing is typically performed for at least about 2 consecutive days (that is, two days in succession) to achieve steady state plasma concentration of ropinirole in the subject. In one embodiment, the dosage form comprises a dose of ropinirole between about 0.5 to about 10 weight percent of ropinirole free base equivalents, wherein the dosage form is a pharmaceutical composition configured for transdermal administration (typically, non-occlusive, transdermal drug delivery).

Embodiments of the present invention also include a dosage form for delivery of ropinirole to a subject comprising, a dose of ropinirole, wherein said dosage form is configured to provide steady-state delivery of ropinirole with once-a-day dosing. The dosage form provides a steady-state oscillation of Cₘₐₓ to Cₘᵢₙ of greater than about 8 hours, more preferably of greater than about 10 hours, and more preferably of greater than about 12 hours, when the subject's plasma level concentration of ropinirole is at steady-state (C_{SS}). The once-a-day dosing is typically performed for at least about 2 consecutive days (that is, two days in succession) to achieve steady state plasma concentration of ropinirole and is continued for the desired course of treatment. In one embodiment, the dosage form comprises a dose of ropinirole between about 0.5 to about 10 weight percent of ropinirole free base equivalents, wherein the dosage form is a pharmaceutical composition configured for transdermal administration (typically, non-occlusive, transdermal drug delivery).

The dosage forms of the present invention can be used, for example, for treatment of a disorder or condition (for example, a movement disorder), as well as for use in preparation of a medicament to treat a disorder or condition.

The present invention provides, in one aspect, a controlled, sustained release of ropinirole over a period of time sufficient to permit a once-a-day dosing. As described above, in one embodiment the dosage form is a composition configured for transdermal application. In other embodiments the dosage form may comprise, for example, ropinirole formulations configured following the guidance of the specification in view of known formulation methods (see, for example U.S. Pat. Nos. 5,156,850, 6,485,746, 6,770,297, 6,861,072, 6,946,146, 6,974,591, 6,987,082, 6,994,871, 7,008,641, and 7,022,339).

These and other objects of the invention will be apparent to one of ordinary skill in the art in view of the teachings presented herein. For example, the concentration of ropinirole in the gel, the amount of gel applied daily, and the surface area over which the gel is applied may be varied by one of ordinary skill in the art in view of the teachings of the present application and the therapeutic needs of the subject being treated.

2.1.0 Exemplary Formulations of the Present Invention and Components Thereof

2.1.1 Transdermal Formulations

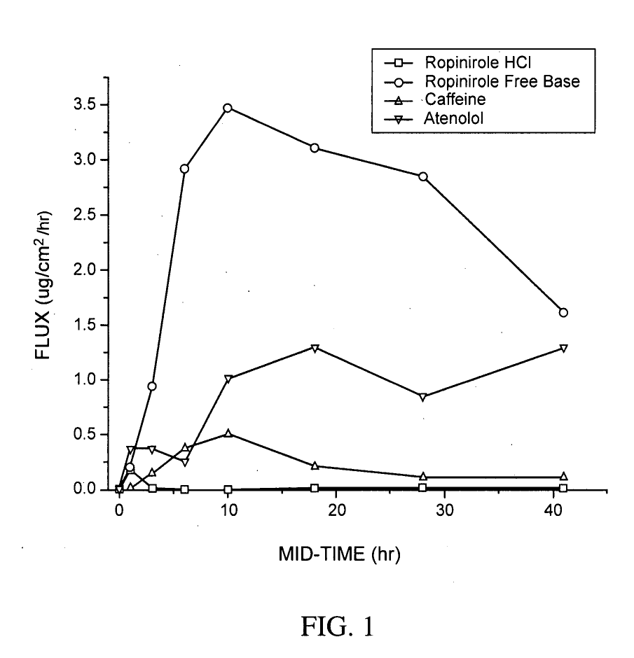

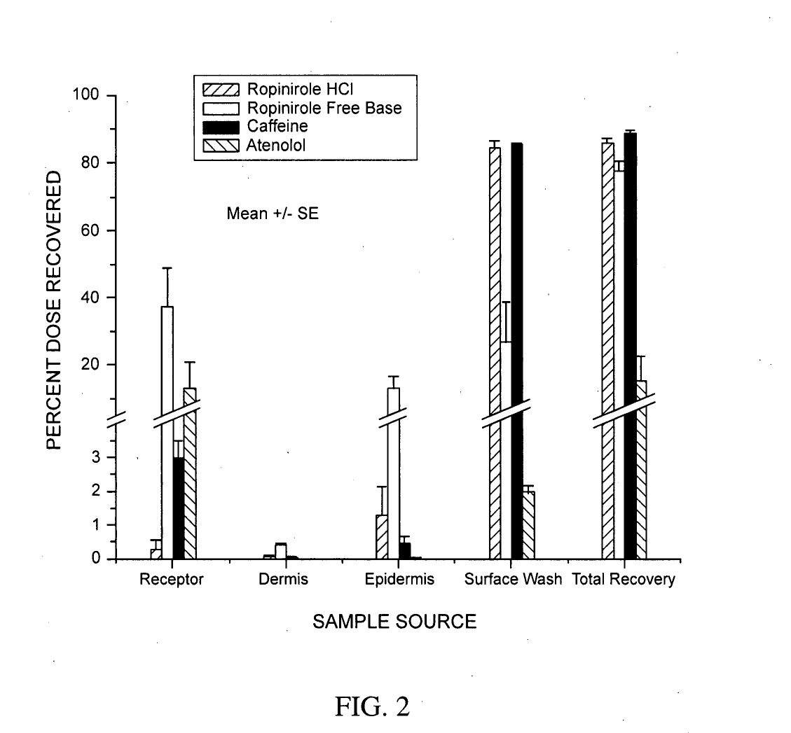

The active ingredient of the formulations of the present invention include indolone compounds and pharmaceutically acceptable salts thereof. A preferred indolone compound is ropinirole, and pharmaceutically acceptable salts thereof. A preferred pharmaceutically acceptable salt of ropinirole is ropinirole HCl. Traditionally, ropinirole has been delivered orally to patients in need of treatment (for example, REQUIP® (SmithKline Beecham, Middlesex UK)). Initial experiments performed in support of the present invention demonstrated that ropinirole free base had good skin permeation characteristics (see, e.g., Example 1; FIG. 1 and FIG. 2). Ropinirole formulations described herein provided sufficient transdermal flux for transdermal gel compositions to be used for therapeutic delivery of ropinirole. In the initial study, a pharmaceutically acceptable salt of ropinirole did not demonstrate skin permeation characteristics in its native substantially protonated form; however, formulation modifications described herein below resulted in excellent permeation characteristics and chemical stability for the pharmaceutically acceptable salt.

In some embodiments, ropinirole was formulated in a hydroalcoholic vehicle. Components of such hydroalcoholic vehicles include, but are not limited to, short-chain alcohols (for example, ethanol, propanol, isopropanol, and/or mixtures of thereof) and water. Typically, the short-chain alcohol(s) and water are considered the primary solvents. Further pharmaceutically acceptable solvents may be included in the formulations as well. In addition, the hydroalcoholic vehicle may include cosolvents, for example, non-volatile cosolvents. Examples of non-volatile solvents include, but are not limited to, propylene glycol, glycerin, liquid polyethylene glycols, polyoxyalkylene glycols, and/or mixtures thereof.

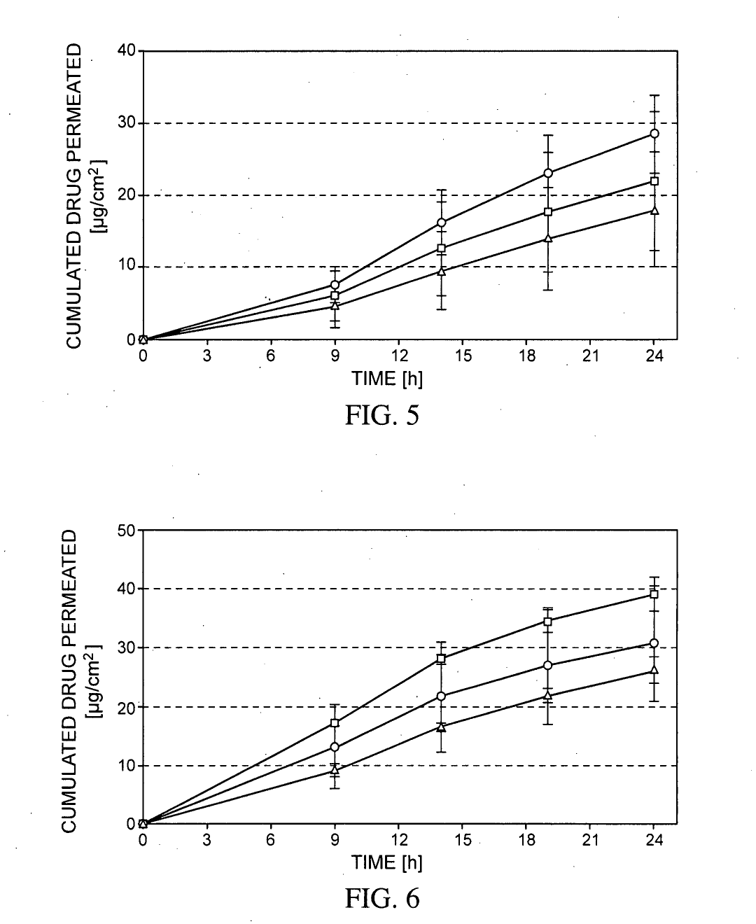

Experiments performed in support of the present invention provided the unexpected result that transdermal permeation of a pharmaceutically acceptable salt of ropinirole (e.g., ropinirole HCl) was sensitive to the concentration of the ropinirole salt in the formulation, when the formulations are at the same pH (see, e.g., Example 4, FIG. 5). The cumulative transdermal permeation of ropinirole in a lower concentration formulation of ropinirole HCl (i.e., 1.7%) was approximately 75% of the transdermal permeation of ropinirole with the higher concentration formulation of ropinirole HCl (i.e., 3.4%). One advantage of obtaining a higher percentage transdermal permeation with pharmaceutically acceptable salts of ropinirole (for example, ropinirole HCl) is the ability to make pharmaceutically efficacious gel formulations using lower concentrations of ropinirole while maintaining the ability to achieve the necessary steady state concentration of ropinirole in the blood of a subject being treated with such gel formulations. Further, the differences in permeation illustrated by the experiments described herein allows flexibility in preparing formulations of ropinirole and pharmaceutically acceptable salts thereof in order to achieve specific, therapeutic, steady-state target ranges for plasma concentrations of ropinirole, for example, by choosing formulation concentrations of ropinirole in the free base form, a pharmaceutically acceptable salt form, or mixtures thereof.

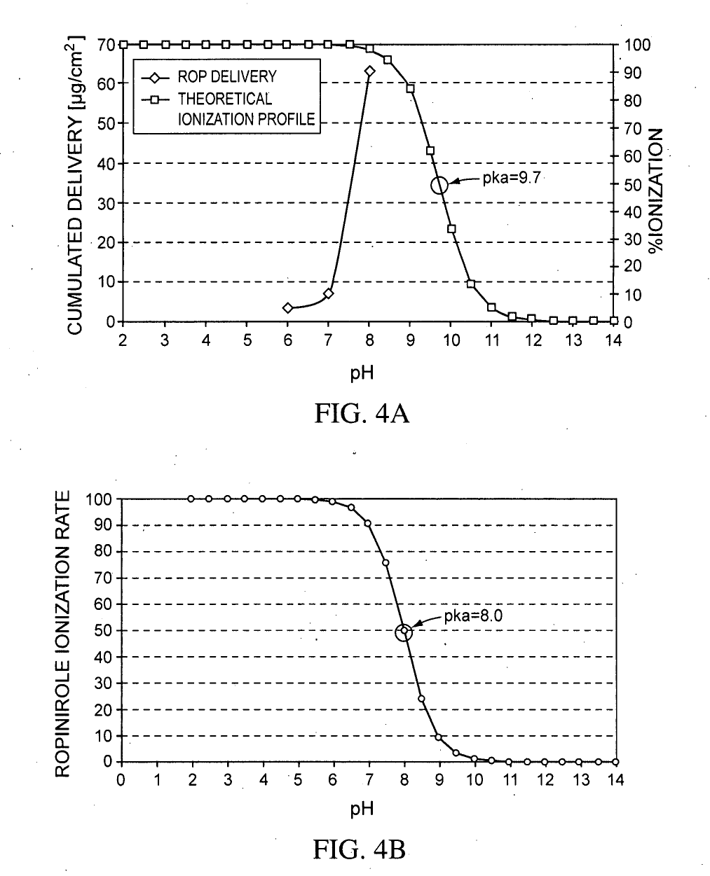

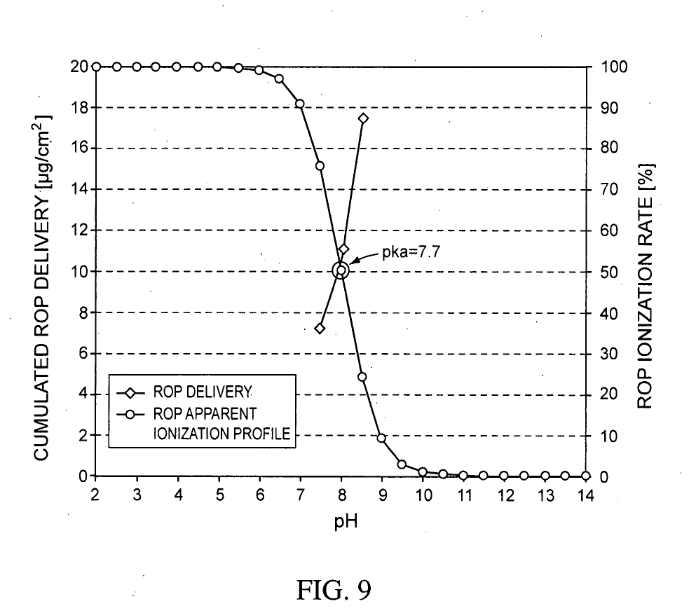

Experiments performed in support of the present invention demonstrated the unexpected finding that the hydroalcoholic vehicle causes an apparent shift in the pKa of ropinirole (see, e.g., Example 3, FIG. 4A, FIG. 4B; Example 6, FIG. 9). The pKa shift in the hydroalcoholic vehicle provides an advantage for formulations of the present invention in that it helps facilitate adjustment of the pH of formulations to pH values closer to the physiological pH of human skin. Another advantage is that the shift of the pKa toward the normal pH range of skin may help reduce the possibility of skin irritation that may be caused by transdermal administration of the formulations of the present invention. Further, the observed pKa shift may help reduce the amount of buffering agent that is added to formulations of ropinirole useful for transdermal applications.

Hydroalcoholic vehicles of the present invention may be gellified, for example, by addition of a gelling agent. Suitable gelling agents of the present invention include, but are not limited to, carbomer, carbomer derivatives, carboxyethylene, polyacrylic acids (for example, Carbopol® (Noveon Ip Holdings Corp. Cleveland, Ohio)), modified cellulose (for example, hydroxypropyl cellulose, hydroxyethyl cellulose, and carboxymethyl cellulose, ethylcellulose, hydroxypropylmethylcellulose, and ethylhydroxyethylcellulose), polyvinyl alcohols, polyvinylpyrrolidone and derivatives, gums (for example, arabic, xanthan, guar gums, carragenans and alginates), and polyoxyethylene polyoxypropylene copolymers. Synonyms for carbopol include carbomer, poly(1-carboxyethylene) and poly(acrylic acid). In view of the teachings of the present specification, one having ordinary skill in the art may identify other gelling agents that are suitable in the practice of the present invention. The gelling agent may, for example, be present from about 1% to about 10% weight to weight of the composition. Preferably, the gelling agent is present from about 0.5% to about 5%, and more preferably, from about 1% to about 3% weight to weight of the composition.

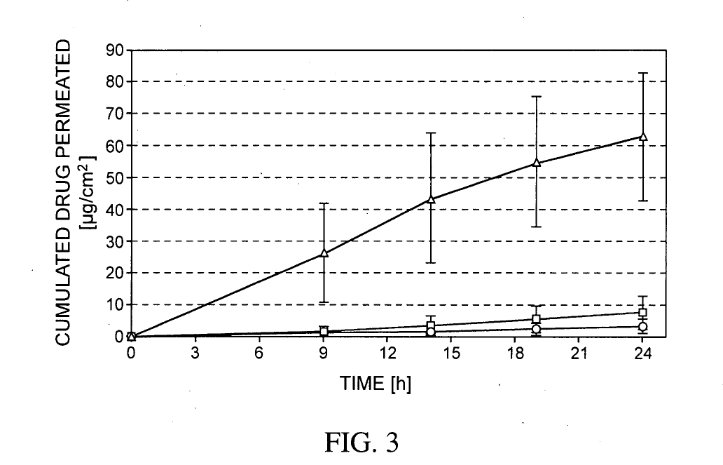

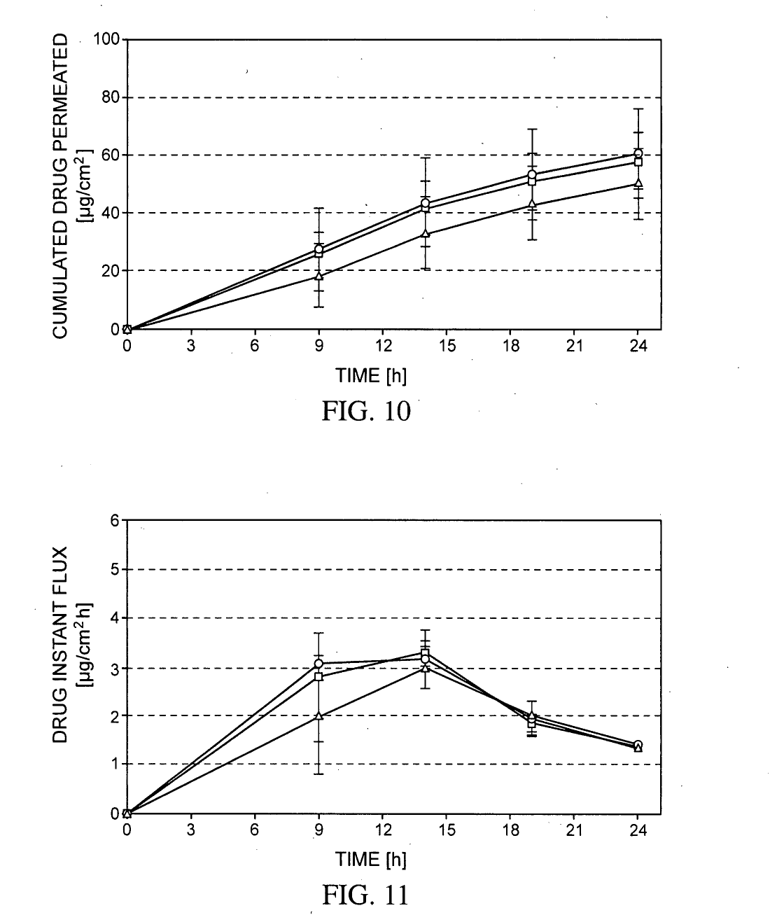

Another unexpected finding obtained from experiments performed in support of the present invention is that (Example 2, FIG. 3; Example 6, FIG. 8, FIG. 9) a large increase in bioavailability of the ropinirole was seen in formulations having pH values of between about pH 7 and about pH 8.5. Thus, it appears desirable to maintain a pH in a target range near the apparent pKa of ropinirole in the hydroalocholic vehicle (that is in the range of about pH 7 to about pH 8.5). Accordingly, the buffering agent (or buffering system) should be able to maintain the pH of the formulation in the target range. After the addition of some buffering agents, further adjustment of pH may be desirable by addition of a second agent to achieve pH values in the target range. In view of the fact that the compositions of the present invention are directed to pharmaceutical use, the buffering agent or system should not be substantially irritating to skin or mucosal tissue to which the composition is being applied. Buffering agents include organic and non-organic buffering agents. Exemplary buffering agents include, but are not limited to, phosphate buffer solutions, carbonate buffers, citrate buffers, phosphate buffers, acetate buffers, sodium hydroxide, hydrochloric acid, lactic acid, tartaric acid, diethylamine, triethylamine, diisopropylamine, diethanolamine, triethanolamine, meglumine and aminomethylamine. Ultimately buffering agents are used at a concentration to achieve the desired target pH range; accordingly weight percent amounts of buffering agents may vary as may be determined by one of ordinary skill in the art in view of the teachings of the present specification. Buffering agents or systems in solution can, for example, replace up to 100% of the water amount within a given formulation. The concentration of a particular buffering agent (pH modifier) did not appear to have a significant effect on permeation and transdermal bioavailability of ropinirole (see, e.g., Example 7, FIG. 10, and FIG. 11).

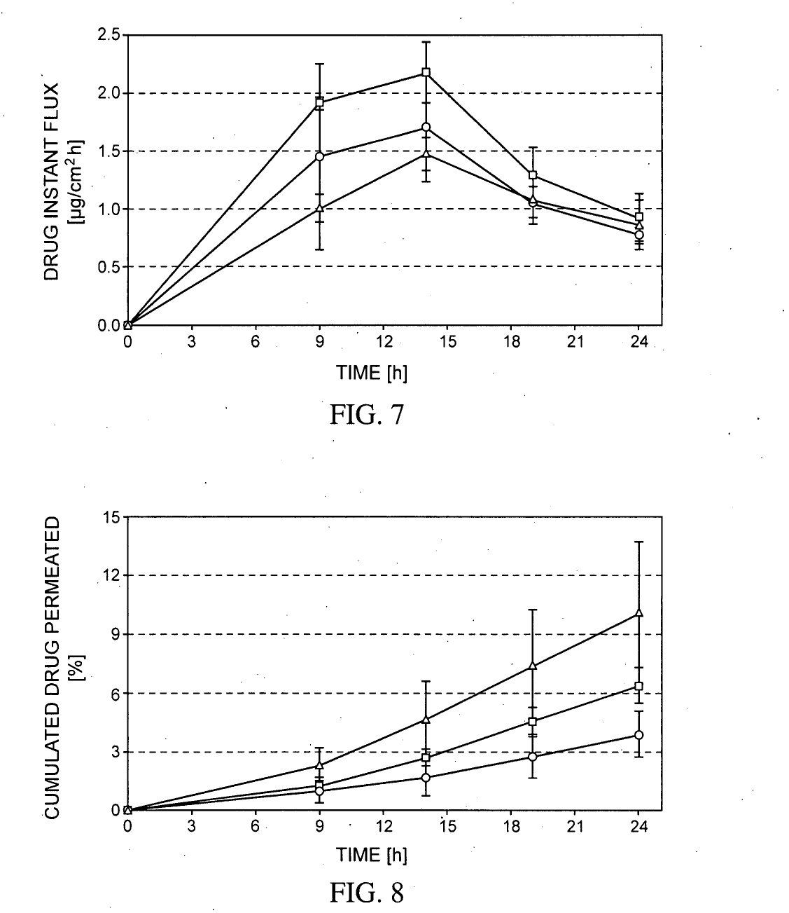

Yet another unexpected result obtained from experiments performed in support of the present invention was that a higher percentage transdermal permeation of ropinirole was seen in the presence of an antioxidant (see, e.g., Example 5, FIG. 6, FIG. 7). The presence of antioxidant (e.g., sodium metabisulfite) enhanced the bioavailability via transdermal permeation of ropinirole. The presence of antioxidants in the formulations of the present invention was also shown to provide stable, pharmaceutically acceptable formulations of ropinirole (see, e.g., Example 9). Exemplary antioxidants include, but are not limited to, tocopherol and derivatives thereof, ascorbic acid and derivatives thereof, butylhydroxyanisole, butylhydroxytoluene, fumaric acid, malic acid, propyl gallate, sodium sulfite, metabisulfites (including sodium metabisulfite) and derivatives thereof, and EDTA disodium, trisodium and the tetrasodium salts. The antioxidant is typically present from about 0.01 to about 5.0% w/w depending on the antioxidant(s) used. As with the other components of the formulations of the present invention, in view of the fact that the compositions are directed to pharmaceutical use, the antioxidant(s) should not be substantially irritating to skin or mucosal tissue to which the composition is being applied.

The compositions of the present invention may further include a permeation enhancer(s). Permeation enhancers are well known in the art (see, for example, U.S. Pat. No. 5,807,570; U.S. Pat. No. 6,929,801; PCT International Publication No. WO 2005/039531; and “Percutaneous Penetration Enhancers”, eds. Smith et al. (CRC Press, 1995)) and may be selected by one of ordinary skill in the art in view of the teachings presented herein for use in the compositions of the present invention. Permeation enhancers include, but are not limited to, sulfoxides, surfactants, fatty alcohols (for example, lauryl alcohol, myristyl alcohol, and oleyl alcohol), fatty acids (for example, lauric acid, oleic acid and valeric acid), fatty acid esters (for example, isopropyl myristate, isopropyl palmitate, methylpropionate, and ethyl oleate), polyols and esters thereof as well as mixtures (for example, propylene glycol, propylene glycol monolaurate), amides and nitrogenous compounds (for example, urea, dimethylacetamide, dimethylformamide, 2-pyrrolidone), and organic acids. The use of an exemplary two-component permeation enhancer (diethylene glycol monoethylether and myristyl alcohol) is described in the formulations set forth in the Examples (see, e.g., Examples 2, 4, 5, 6, and 7). PCT International Publication No. WO 2005/039531 describes the combined use, preferably in hydroalcoholic vehicles, of a monoalkyl ether of diethylene glycol and a glycol in specific ratios as permeation enhancers.

Further amphiphilic and non-amphiphilic molecules may be used as penetration enhancers. Amphiphilic molecules are characterized as having a polar water-soluble group attached to a water-insoluble hydrocarbon chain. In general, amphiphilic penetration enhancers have a polar head group and long aliphatic tail. These categories include: surfactants, short chain alcohols, organic acids, charged quaternary ammonium compounds. Examples of such amphiphilic solvents are butanediols, such as 1,3-butanediol, dipropylene glycol, tetrahydrofurfuryl alcohol, diethylene glycol dimethyl ether, diethylene glycol monoethylether, diethylene glycol monobutyl ether, propylene glycol, dipropylene glycol, carboxylic acid esters of tri- and diethylene glycol, polyethoxylated fatty alcohols of 6-18 C atoms or 2,2-dimethyl-4-hydroxymethyl-1,3-dioxolane (Solketal®) or mixtures of these solvents.

Without intending to be bound by any specific theory of operation, non-amphiphilic penetration enhancers are believed to operate by “shunting” the drug substance through pores, sweat glands and hair follicles, and opening the intercellular spaces of the stratum corneum, among other ways (Asbill et al., 2000, “Enhancement of transdermal drug delivery: chemical and physical approaches, “Crit Rev Ther Drug Carrier Syst, 17:621-58). Regarding the latter, the proteinaceous intracellular matrices of the stratum corneum, together with the diverse biochemical environments of the intercellular domains in the stratum corneum, represent a formidable barrier to drugs before they can reach the deeper parts of epidermis (e.g., the stratum germinativum) and dermis. Once absorbed into the stratum corneum, effects of the non-amphiphilic penetration enhancer may include altering the solvent potential of the stratum corneum biochemical environment (i.e., the ability of stratum corneum to retain drug substances in a non-crystalline form), and disordering the ordered structure of the intercellular lipid region (for example, due to insertion of the non-amphiphilic penetration enhancer molecule between the parallel carbon chains of the fatty acids). For illustration and not limitation, exemplary non-amphiphilic penetration enhancers are: 1-menthone, isopropyl myristate, dimethyl isosorbide, caprylic alcohol, lauryl alcohol, oleyl alcohol, isopropyl butyrate, isopropyl hexanoate, butyl acetate, methyl acetate, methyl valerate, ethyl oleate, d-piperitone, d-pulogene, n-hexane, citric acid, ethanol, propanol, isopropanol, ethyl acetate, methyl propionate, methanol, butanol, tert-butanol, octanol, myristyl alcohol, methyl nonenoyl alcohol, cetyl alcohol, cetearyl alcohol, stearyl alcohol, myristic acid, stearic acid, and isopropyl palmitate.

Other non-amphiphilic penetration enhancers can be identified using routine assays, e.g., in vitro skin permeation studies on rat, pig or human skin using Franz diffusion cells (see Franz et a., “Transdermal Delivery” In: Treatise on controlled Drug Delivery. A. Kydonieus. Ed. Marcell Dekker: New York, 1992; pp 341-421). Many other methods for evaluation of enhancers are known in the art, including the high throughput methods of Karande and Mitragotri, 2002, “High throughput screening of transdermal formulations” Pharm Res 19:655-60, and Karande and Mitragotri, 2004, “Discovery of transdermal penetration enhancers by high-throughput screening”).

Non-amphiphilic penetration enhancers suitable for use in the present invention are pharmaceutically acceptable non-amphiphilic penetration enhancers. A pharmaceutically acceptable non-amphiphilic penetration enhancer can be applied to the skin of a human patient without detrimental effects (i.e., has low or acceptable toxicity at the levels used).

Non-amphiphilic penetration enhancers suitable for use with the methods and devices described here include, but are not limited to, enhancers from any of the following classes: fatty long chain alcohols, fatty acids (linear or branched); terpenes (e.g., mono, di and sequiterpenes; hydrocarbons, alcohols, ketones); fatty acid esters, ethers, amides, amines, hydrocarbons, alcohols, phenols, polyols.

The amount of permeation enhancer present in the composition will depend on a number of factors, for example, the strength of the permeation enhancer, the desired increase in skin permeability, the amount of drug to be delivered, the solubility of the drug in the matrix and the desired rate of administration. The effects of permeation enhancers in the compositions of the present invention can be evaluated by one of ordinary skill in the art following the teachings of the present specification (see, e.g., description of permeation study methods in the Materials and Methods section, herein below). Preferred ranges of permeation enhancer(s) in the compositions of the present invention are generally between about 0.1% and about 10% (w/w).

Example 8 (Table 14) sets forth general formulation guidelines for some embodiments of gels for application to the skin surface of a subject in need of ropinirole therapy. In these formulations, the primary vehicle of the transdermal gel formulations is a gellified hydroalcoholic mixture (e.g., ethanol/water gellified with hydroxypropyl cellulose). The transdermal gel formulations of the present invention contain a pharmaceutically effective amount of active drug (e.g., ropinirole), and typically have a final pH of between about 7.0 and about 9.0, more preferably between about 7.0 and about 8.5, more preferably between about 7.5 and about 8.5.

Although preferred general components of the compositions of the present invention are described herein above, additional components may be included by one of ordinary skill in the art in view of the teachings presented herein. Further components may include, but are not limited to, humectants, moisturizers, surfactants, fragrances, and emollients.

In one aspect, the present invention relates to a gel formulation of ropinirole that is able to deliver ropinirole via transdermal application to a subject and achieve systemic absorption rates comparable or superior to oral tablets of ropinirole. In some embodiments, the present invention describes the use of a combination of permeation enhancers to achieve sustained transdermal delivery of ropinirole. Typically, the excipients and permeation enhancers used in the formulations of the present invention are either compendial or CFR listed; accordingly, no specific toxicity studies are required. The gel formulations of the present invention suitable for transdermal use represent an alternative to oral tablet dosing. Such formulations provide the advantages of delivering constant, sustained and smoothed plasmatic levels of ropinirole while offering dose regimen flexibility (e.g., once a day dosing versus oral tablets every eight hours). Further, the gel formulations of the present invention provide an alternative route of administration for ropinirole for subjects in need thereof, for example, geriatric patients who are often poly-medicated and sometimes have difficulty swallowing oral dosage forms. The gel formulations of the present invention can be provided for use in unit-dose packaging (for example, airless metered-dose pumps or single use pouches) to ease administration and ensure correct dosing for subjects.

Further, although preferred methods of administration are described herein (for example, gel compositions for application to skin surface), the compositions of the present invention are broadly suitable for use in transdermal applications (for example, intranasal delivery or delivery by suppository) as can be determined by one of ordinary skill in the art in view of the teachings presented herein.

### Further Dosage Forms

As described above, the present invention provides a dosage form comprised of a desired dose of ropinirole, where the dosage form provides sustained release of ropinirole. In general, the dosage form provides for the delivery of ropinirole over a prolonged period of time such that once-a-day administration of the drug is possible. The dosage form may also deliver ropinirole in a manner that results in relatively fewer and/or reduced side affects (for example, gastrointestinal side effects).

A simulated ropinirole delivery profile for an exemplary transdermal dosage form of the present invention is illustrated in FIG. 13. FIG. 13, shows predicted plasma concentration over a one week period for a ropinirole transdermal administration for 5 consecutive days. The predicted plasma concentration was obtained by simulation for administration of 0.2 g of gel at 3.4% ropinirole HCl strength applied over 35 cm² skin area once per day. The simulation is based on the assumption (from in vitro human skin penetration studies) that there are two input phases: the first burst, having a faster flux rate of 4.5 μg/cm²/hr and, the second maintenance, having a slower flux rate of 2.75 μg/cm²/hr. The data in the figure show, at steady state, a Cₘₐₓ of about 5.2 ng/ml, a Cmin of about 4.1 ng/ml, and a C_{SS} of about 4.6 ng/ml. The Cₘₐₓ/Cₘᵢₙ ratio at steady state is about 1.27. Further, the total time at steady-state of the oscillation of Cₘₐₓ to Cₘᵢₙ in FIG. 13 is about 15 hours and the Cₘᵢₙ to Cₘₐₓ is about 9 hours.

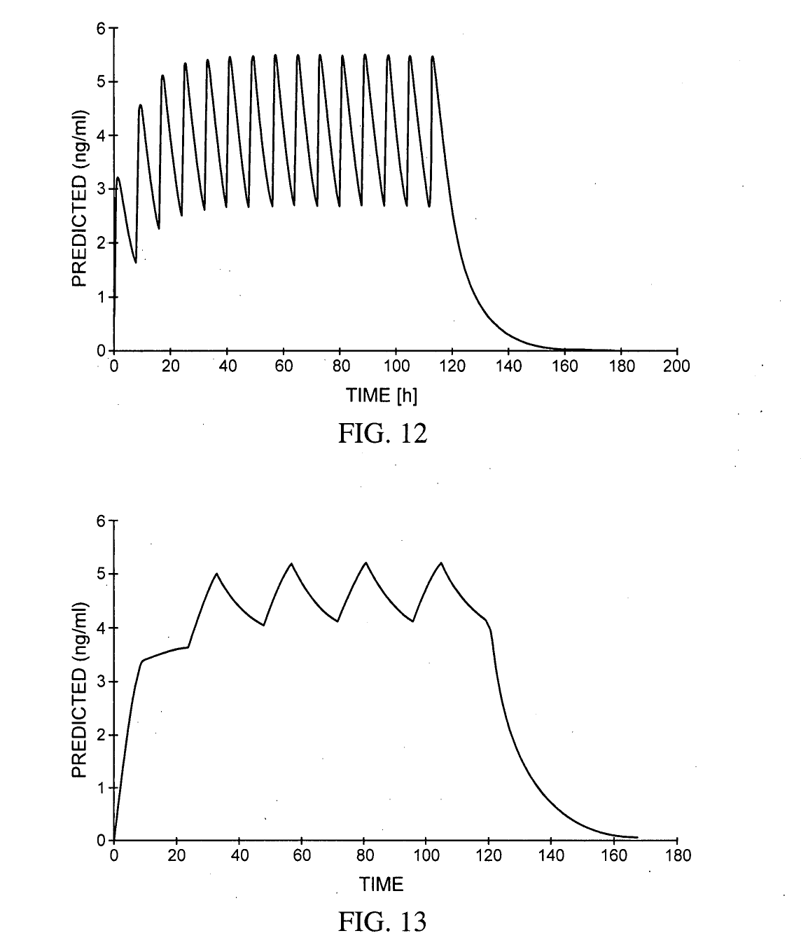

This example of a ropinirole delivery profile for a dosage form of the present invention can be compared to the predicted plasma concentration over a one week period for a standard oral dosage form of ropinirole delivered by oral administration for 5 consecutive days. The predicted plasma concentration presented in FIG. 12 was obtained by simulation of administration of a 2 mg tablet of ropinirole given every 8 hours (i.e., three times a day). The data in the figure show, at steady sate, a Cₘₐₓ of about 5.5 ng/ml, a Cₘᵢₙ of about 2.7 ng/ml, and a C_{SS} of about 4.1 ng/ml. The Cₘₐₓ/Cₘᵢₙ ratio for this oral dosage form, about 2.04, is relatively higher than the Cₘₐₓ/Cₘᵢₙ ratio for the dosage form of the present invention shown in FIG. 13. Further, the steady-state oscillation of Cₘₐₓ to Cₘᵢₙ, in FIG. 12 is about 6.5 hours and the Cₘᵢₙ to Cₘₐₓ is about 1.5 hours. Accordingly, the steady-state oscillation of Cₘₐₓ to Cₘᵢₙ is relatively faster in the standard oral dosage form than in the dosage form of the present invention as shown above in FIG. 13.

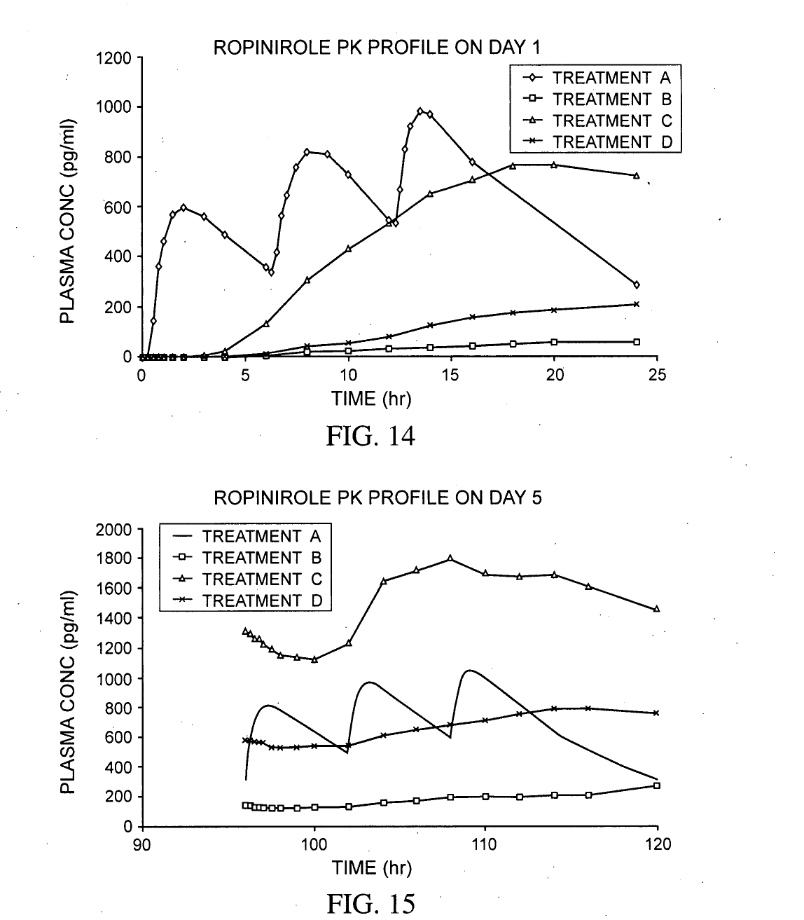

From the foregoing simulated delivery profiles and the actual pharmacokinetic profiles shown in FIGS. 14 and 15 and described in Example 12, it is apparent that the invention provides a dosage form with a profile that permits once daily dosing of ropinirole. The profiles shown in FIGS. 13 and 15 provides a once-a-day dosage form where (i) a steady-state ratio of Cₘₐₓ/Cₘᵢₙ that is less than about 1.75, more preferably less than about 1.5, and more preferably less than about 1.3 when the subject's plasma level concentration of ropinirole is at steady-state; (ii) a steady-state oscillation of Cₘₐₓ to Cₘᵢₙ of greater than about 8 hours, more preferably greater than 10 hours, and more preferably greater than 12 hours, when the subject's plasma level concentration of ropinirole is at steady-state; and (iii) a steady-state oscillation of Cₘᵢₙ to Cₘₐₓ of less than about 9 hours. The sustained release dosage forms of the present invention provide controlled delivery of therapeutically effective concentrations of ropinirole over prolonged periods of time using, for example, once-a-day dosing.

Further, although preferred dosage forms are described herein, further dosage forms of the compositions of the present invention can be determined by one of ordinary skill in the art in view of the teachings presented herein.

2.2.0 Manufacturing and Packaging

Exemplary methods of making or manufacturing the compositions of the present invention are described herein below in the Materials and Methods section. Variations on the methods of making the compositions of the present invention will be clear to one of ordinary skill in the art in view of the teachings contained herein.

The manufacturing process for gel formulations of the present invention is straightforward and is typically carried out in a closed container with appropriate mixing equipment. For example, ethanol, propylene glycol, diethylene glycol monoethylether, and myristyl alcohol are mixed in a primary container (reaction vessel) under a slight vacuum and nitrogen blanketing until a clear solution forms. Methods of degassing the solvents may include nitrogen sparge of the application of vacuum. In parallel, sodium metabisulfite is dissolved in a portion of water in a separate container and then added to the primary solution to prepare a hydroalcoholic solution. Ropinirole is added to the hydro-alcoholic solution. The pH is then brought to its final value (e.g., approximately pH 8.0) by adding a fixed amount of triethanolamine. The solution is gellified by addition of hydroxypropylcellulose and is then stirred until the hydroxypropylcellulose is completely swollen.

The compositions of the present invention may be applied to a skin surface or mucosal membrane using a variety of means, including, but not limited to a pump-pack, a brush, a swab, a finger, a hand, a spray device or other applicator.

The methods of manufacturing of the present invention may include dispensing compositions of the present invention into appropriate containers. The compositions of the present invention may be packaged, for example, in unit dose or multi-dose containers. The container typically defines an inner surface that contains the composition. Any suitable container may be used. The inner surface of the container may further comprise a liner or be treated to protect the container surface and/or to protect the composition from adverse affects that may arise from the composition being in contact with the inner surface of the container. Exemplary liners or coating materials include, but are not limited to high density polyethylene, low density polyethylene, very low density polyethylene, polyethylene copolymers, thermoplastic elastomers, silicon elastomers, polyurethane, polypropylene, polyethylene terephthalate, nylon, flexible polyvinylchloride, natural rubber, synthetic rubber, and combinations thereof. Liners or coating material are typically substantially impermeable to the composition and typically to the individual components of the composition.

A number of types of containers are known in the art, for example, packets with rupturable barriers (see, for example, U.S. Pat. Nos. 3,913,789, 4,759,472, 4,872,556, 4,890,744, 5,131,760, and 6,379,069), single-use packets (see, for example, U.S. Pat. Nos. 6,228,375, and 6,360,916), tortuous path seals (see, for example, U.S. Pat. Nos. 2,707,581, 4,491,245, 5,018,646, and 5,839,609), and various sealing valves (see, for example, U.S. Pat. Nos. 3,184,121, 3,278,085, 3,635,376, 4,328,912, 5,529,224, and 6,244,468). One example of a unit dose container is a flexible, foil packet with a polyethylene liner.

Containers/Delivery systems for the compositions of the present invention may also include a multi-dose container providing, for example a fixed or variable metered dose application. Multi-dose containers include, but are not limited to, a metered dose aerosol, a stored-energy metered dose pump, or a manual metered dose pump. In preferred embodiments, the container/delivery system is used to deliver metered doses of the compositions of the present invention for application to the skin of a subject. Metered dose containers may comprise, for example, an actuator nozzle that accurately controls the amount and/or uniformity of the dose applied. The delivery system may be propelled by, for example, a pump pack or by use of propellants (e.g., hydrocarbons, hydro-fluorocarbons, nitrogen, nitrous oxide, or carbon dioxide). Preferred propellants include those of the hydrofluorocarbon (e.g., hydrofluoroalkanes) family, which are considered more environmentally friendly than the chlorofluorocarbons. Exemplary hydrofluoroalkanes include, but are not limited to, 1,1,1,2-tetrafluoroethane (HFC-134(a)), 1,1,1,2,3,3,3,-heptafluoropropane (HFC-227), difluoromethane (HFC-32), 1,1,1-trifluoroethane (HFC-143(a)), 1,1,2,2-tetrafluoroethane (HFC-134), 1,1-difluoroethane (HFC-152a), as well as combinations thereof. Particularly preferred are 1,1,1,2-tetrafluoroethane (HFC-134(a)), 1,1,1,2,3,3,3,-heptafluoropropane (HFC-227), and combinations thereof. Many pharmaceutically acceptable propellants have been previously described and may be used in the practice of the present invention in view of the teachings presented herein. The delivery system should provide dose uniformity. In a preferred embodiment, airless packaging with excellent barrier properties is used to prevent oxidation of ropinirole, for example, airless metered-dose pumps wherein the composition comprising ropinirole is packaged in collapsible aluminum foils. Accurate dosing from such pumps ensures reproducibility of dose.

Uses of the Formulations of the Present Invention

The present invention further includes methods for administering a composition of the present invention to a subject in need thereof. Compositions of the present invention comprising ropinirole can be employed, for example, for the treatment of a variety of conditions and/or disease states which have been historically treated by oral doses of ropinirole (for example, using REQUIP®). Ropinirole therapy has been used to treat a variety of diseases and disorders of the central nervous system, including movement disorders (see, for example, U.S. Pat. Nos. 4,824,860, 5,807,570, and 6,929,801; and “Clinical Pharmacokinetics of Ropinirole,” by C. M. Kaye, et al., Clin. Pharmacokinet. 39(4):2443-254 (2000)). Some specific conditions/disease states responsive to treatment with ropinirole include, but are not limited to, Parkinson's Disease, Restless Legs Syndrome, Tourette's Syndrome, Chronic Tic Disorder, Essential Tremor, and Attention Deficit Hyperactivity Disorder.

The ropinirole compositions of the present invention may be self-applied by a subject in need of treatment or the composition may be applied by a care-giver or health care professional. The compositions may be applied in single daily doses, multiple daily doses, or divided doses. Transdermal delivery of ropinirole, as described herein, provides a number of advantages relative to oral dosing, including, but not limited to, continuous delivery which provides for steady-state blood levels of the ropinirole, avoidance of the first-pass effect, and substantial avoidance of gastrointestinal and many other side effects. The likelihood of patient acceptance may also be much improved particularly among populations that have difficulty swallowing pills, for example, some elderly subjects. In view of the data presented in the Example 13, herein below, skin irritation arising from use of the compositions of the present invention is likely to be minimal.

Ease of application of the compositions of the present invention, for example, gel formulations comprising ropinirole, provides several advantages relative to oral administration of ropinirole. For example, when the subject in need of treatment cannot self-medicate (e.g., young children or the infirmed) transdermal delivery avoids forcing subjects to take and swallow a pill. Further, transdermal application of the compositions of the present invention assures correct dosing, versus a pill that may be inappropriately chewed (for example, when the pill is a time-release formulation), spit out, and/or regurgitated. Dose escalation or titration is particularly facilitated by a ropinirole transdermal gel in that larger doses may be administered by increasing the area of application to the skin while keeping the concentration of the formulation fixed.

In one embodiment of the present invention, up to about 1.0 grams of a gel formulation, having an amount of ropinirole free base equivalents between about 3 and about 5 weight percent, is applied daily to a skin surface area of between about 50 to about 1,000 cm². In another embodiment, up to about 0.5 grams of a gel formulation, having an amount of ropinirole free base equivalents of about 1.5 weight percent, is applied daily to a skin surface area of between about 70 to about 500 cm². In yet another embodiment, the composition is a set that has an amount of ropinirole free base equivalents of about 3.0 weight percent, where 0.25 grams of gel is applied to a skin surface area of about 50 to 300 cm².

Experiments performed in support of the present invention have provided good in vitro/in vivo correlation based on bioavailability of ropinirole in the compositions of the present invention. These results are intended for illustration purposes only and to provide a general basis for in vitro/in vivo comparison, thus they should not be considered limiting. As a first example, in vitro/in vivo correlation based on bioavailability of Formulation C1 (Example 2; 3% ropinirole free-base equivalents) may be evaluated as follows. In vitro data can be extrapolated to in vivo conditions in order to evaluate the gel dose for bioequivalence to ropinirole oral absorption. REQUIP® tablets are typically administered at doses ranging between 3-9 mg per day, with an oral bioavailability (BA) of 50% (see, for example, REQUIP® Prescribing Information, GlaxoSmithKline, Middlesex UK). Therefore, an intermediate oral dose of 6 mg/day with BA=50% delivers a systemic dose of 3 mg/day. Considering that Formulation C1 has a transdermal bioavailability of about 36%, Formulation C should be bioequivalent to the 6 mg oral dose (3 mg systemic dose) if 0.3 g of the Formulation C1 gel is applied over about 53 cm² of skin surface. This corresponds to a daily dose of 9.5 mg ropinirole HCl (equivalent to 8.3 mg free base).

Taylor, et al., (“Lack of a Pharmacokinetic Interaction at Steady State Between Ropinirole and L-Dopa in Patients With Parkinson's Disease,” Pharmacotherapy 19(2):150-156 (1999)) have shown that repeated oral administration of ropinirole (6 mg/day in 3 divided doses) generated maximum plasma levels (Cₘₐₓ) of 7.4 ng/mL. Body clearance of ropinirole is about 47 L/h (see, for example, REQUIP® Prescribing Information, GlaxoSmithKline, Middlesex UK). Based on these pharmacokinetic parameters, the daily input rate can be estimated using the following equation: Kₐ=CL×Cₚ, where Kₐ is the daily input rate (absorption rate), CL the drug plasma clearance, and Cₚ the plasma concentration. Thus, the Kₐ for Ropinirole is 347.8 μg/h.

Scaled to the clinical daily input rate, the required skin surface can be determined using the following equation: S=Kₐ/J_{SS}, wherein S is the application skin surface area, and J_{SS} is the in vitro steady-state drug flux. In the present example, J_{SS}=1.9 μg/cm²h for Formulation C1 corresponding, therefore, to a surface area of 183 cm², which is 3.5 times higher than what is predicted from the in vitro transdermal bioavailability. However, it should be noted that the in vitro ropinirole flux used in these calculations was observed for a single application, and was therefore probably underestimated—repeated application likely provides higher levels.

Alternatively, steady-state plasma levels of Formulation C1 may be predicted using the steady-state in vitro flux, the assumed skin application surface, and ropinirole clearance, according to following equation: C_{SS}=J_{SS}×S/CL, wherein C_{SS} is the plasma level at steady state, J_{SS} the in vitro flux at steady-state, S the skin application surface area, and CL the drug plasma clearance. Using an in vitro steady-state flux of 1.9 μg/cm²h, and a clearance of 47 L/h, it can be estimated that transdermal application of Formulation C1 over 50 cm² skin should be able to attain and maintain 2 ng/mL over a period of one day, after single dose application. This level is 3.7 times lower than the Cₘₐₓ observed by Taylor, et al., (cited above), which was 7.4 ng/mL after repeated oral administration of ropinirole at steady-state (6 mg/day in 3 divided doses). However, C_{SS} are always lower than Cₘₐₓ, and the theoretical plasma level is likely underestimated. Repeated daily application of the gel Formulation C1 should theoretically result in similar Cₘₐₓ as for oral administration. Alternatively, the gel amount could be increased by 3.7 times (1 g instead of 0.3 g), and be applied to a 3.7 times larger skin area (185 cm instead of 50 cm²).

In one embodiment of the present invention, 5 g of a gel formulation of ropinirole at 3-5% (ropinirole free-base equivalents) is applied over 50-500 cm² of skin surface. These results generally demonstrate the feasibility of the transdermal ropinirole delivery using a gel formulation of the present invention, because, for example, Formulation C1 is at 3.4% HCl salt strength (equivalent to 3% free base), and was estimated to be bioequivalent to oral tablets if about 0.3-1 g of gel (containing 10-34 mg ropinirole HCl, corresponding to 9-30 mg free base) are topically applied over a skin area of about 50-185 cm².

As a second example, in vitro/in vivo correlation based on bioavailability of Formulation B2 (Example 4; 1.5% ropinirole free-base equivalents) was evaluated essentially as described above. With transdermal bioavailability of about 23%, Formulation B2 should be bioequivalent to the 6 mg oral dose (3 mg systemic dose) if 0.9 g of the gel Formulation B2 is applied topically over 160 cm² skin. This corresponds to a daily dose of 15 mg ropinirole HCl (equivalent to 13 mg free base). Applying the same methodology as described above, and using the steady-state in vitro flux of Formulation B2 (0.94 μg/cm²/h), the theoretical skin application surface for generating ropinirole peak plasma levels of 7.4 ng/mL is 370 cm². In this example, the bioequivalent surface area was 160 cm², which is 2.3 times lower than what is predicted from peak plasma levels. However, it should be noted that the in vitro ropinirole flux used in these calculations was observed for a single application, and was therefore probably underestimated—repeated application likely provide higher levels.

Alternatively, plasma levels of Formulation B2 can be predicted using the steady-state in vitro flux, as described above. With an in vitro steady-state flux of 0.94 μg/cm²/h, and a clearance of 47 L/h, it can be estimated that application of Formulation B2 over 160 cm² skin should be able to attain and maintain 3.2 ng/mL over a period of one day, after single dose application. This level is 2.3 times lower than the Cₘₐₓ observed by Taylor, et al., (cited above), which was 7.4 ng/mL after repeated oral administration of ropinirole at steady-state (6 mg/day in 3 divided doses). Again, C_{SS} are always lower than Cₘₐₓ, and the theoretical plasma level is likely underestimated. Repeated daily application of the gel of Formulation B2 should theoretically result in similar Cₘₐₓ as for oral administration. Alternatively, the amount of gel of Formulation B2 could be increased by 2.3 times (2 g instead of 0.9 g), and be applied to a 2.3 times larger skin area (370 cm² instead of 160 cm²).

This example further illustrates the feasibility of the transdermal ropinirole delivery by the compositions of the present invention, for example, Formulation B2, because formulation B2 was at 1.7% HCl salt strength (equivalent to 1.5% free base), and was estimated to be bioequivalent to oral tablets if about 0.9-2 g of gel (containing 15-34 mg ropinirole) are applied over a skin area of 160-370 cm². Formulation B2 illustrates a good compromise formulation between drug strength and transdermal delivery.

Theoretical evaluations of transdermal ropinirole delivery using exemplary compositions of the present invention have shown the feasibility to achieve therapeutic levels, for example, application of 0.9-2 g of gel at 1.7% Ropinirole HCl (equivalent to 1.5% free base) over 160-370 cm² skin surface theoretically provides similar plasma levels as an intermediate 6 mg oral dose of REQUIP®.

Because theoretical predictions of gel amount and skin application area from in vitro data may be underestimated, the formulations of the present invention may be tested in a clinical setting for determination of actual dosing requirements for selected formulations of the present invention, for example, as discussed in Example 11 and further tested in Example 12. Exact dosing requirements may be determined by one of ordinary skill in the art, for example, a research physician, in view of the teachings of the present specification. Further, such clinical testing provides information concerning therapeutic effectiveness of the ropinirole formulations of the present invention for the treatment of a variety of conditions/disease states, as well as information regarding side-effects.

The following examples are illustrative of embodiments of the present invention and should not be interpreted as limiting the scope of the invention.

Experimental

The following examples are put forth so as to provide those of ordinary skill in the art with a complete disclosure and description of how to make and use the formulations, methods, and devices of the present invention, and are not intended to limit the scope of what the inventors regard as the invention. Efforts have been made to ensure accuracy with respect to numbers used (e.g., amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

The compositions produced according to the present invention meet the strict specifications for content and purity required of pharmaceutical products.

Materials and Methods

Pharmaceuticals and Reagents.

The pharmaceuticals and reagents used in the following examples can be obtained from commercial sources, for example, as follows: active drug (e.g., ropinirole (free-base form and ropinirole hydrochloride, from PCAS, Oy, Finland); penetration enhancers (e.g., diethylene glycol monoethylether, also called TRANSCUTOL®P, from Gattefossé Corporation, Paramus, N.J.; urea, myristyl alcohol, from Sigma-Aldrich Corporation, St. Louis, Mo.); solvents and cosolvents (e.g., ethanol, propylene glycol, from Sigma-Aldrich Corporation, St. Louis, Mo.); antioxidants (e.g., butylhydroxytoluene (BHT), butylhydroxyanisole (BHA), sodium metabisulfite, from Sigma-Aldrich Corporation, St. Louis, Mo.); thickening or gelling agents (e.g., hydroxypropyl cellulose, from Sigma-Aldrich Corporation, St. Louis, Mo.; or KLUCEL® (Aqualon Company, Wilmington Del.) hydroxypropyl cellulose, from Hercules, Inc., Wilmington, Del.); and standard pharmaceutical and chemical reagents (e.g., triethanolamine, sodium hydroxide, from Sigma-Aldrich Corporation, St. Louis, Mo.).

In Vitro Skin Permeation Methodology.

The in vitro human cadaver skin model has proven to be a valuable tool for the study of percutaneous absorption and the determination of topically applied drugs. The model uses human cadaver skin mounted in specially designed diffusion cells that allow the skin to be maintained at a temperature and humidity that match typical in vivo conditions (Franz, T. J., “Percutaneous absorption: on the relevance of in vitro data,” J. Invest Dermatol 64:190-195 (1975)). A finite dose (for example: 4-7 mg/cm²) of formulation is applied to the outer surface of the skin and drug absorption is measured by monitoring its rate of appearance in the receptor solution bathing the inner surface of the skin. Data defining total absorption, rate of absorption, as well as skin content can be accurately determined in this model. The method has historic precedent for accurately predicting in vivo percutaneous absorption kinetics (Franz, T. J., “The finite dose technique as a valid in vitro model for the study of percutaneous absorption in man,” In: Skin: Drug Application and Evaluation of Environmental Hazards, Current Problems in Dermatology, vol. 7, G. Simon, Z. Paster, M Klingberg, M. Kaye (Eds), Basel, Switzerland, S. Karger, pages 58-68 (1978)).

Pig skin has been found to have similar morphological and functional characteristics as human skin (Simon, G. A., et al., “The pig as an experimental animal model of percutaneous permeation in man,” Skin Pharmacol. Appl. Skin Physiol. 13(5):229-34 (2000)), as well as close permeability character to human skin (Andega, S., et al., “Comparison of the effect of fatty alcohols on the permeation of melatonin between porcine and human skin,” J. Control Release 77(1-2):17-25 (2001); Singh, S., et al., “In vitro permeability and binding of hydrocarbons in pig ear and human abdominal skin,” Drug Chem. Toxicol. 25(1):83-92 (2002); Schmook, F. P., et al., “Comparison of human skin or epidermis models with human and animal skin in in vitro percutaneous absorption,” Int. J. Pharm. 215(1-2):51-6 (2001)). Accordingly, pig skin may be used for preliminary development studies and human skin used for final permeation studies. Pig skin can be prepared essentially as described below for human skin.

Skin Preparation.

Percutaneous absorption was measured using the in vitro cadaver skin finite dose technique. Cryo-preserved, human cadaver trunk skin was obtained from a skin bank and stored in water-impermeable plastic bags at <−70° C. until used.

Prior to the experiment, skin was removed from the bag, placed in approximately 37° C. water for five minutes, and then cut into sections large enough to fit on 1 cm² Franz Cells (Crown Glass Co., Somerville, N.J.). Briefly, skin samples were prepared as follows. A small volume of phosphate buffered saline (PBS) was used to cover the bottom of the Petri dishes. Skin disks generally depleted of fat layers were placed in the Petri dishes for hydration. A Stadie-Riggs manual tissue microtome was used for slicing excised skin samples. Approximately 2 mL of PBS was placed into the middle cavity of the microtome as slicing lubricant. Skin disks were placed, dermal side up, into the middle cavity of the microtome. Filter paper was soaked with PBS, inserted in the cavity just above the skin disk. The filter paper prevented the dermis from sliding onto the top of the cutting block and helped to insure more precise cutting. When all three blades of the microtome were assembled, the microtome was turned into the upright position. Using a regular and careful sawing motion the skin tissue was sliced in cross-section. The skin tissue slice was removed with the tweezers and placed in the Petri dish for hydration. Each skin slice was wrapped in Parafilm® (Pechiney Plastic Packaging, Inc., Chicago, Ill.) laboratory film and placed in water-impermeable plastic bags. Skin samples were identified by the donor and the provider code. If further storage was necessary, the skin slices were stored in the freezer at −20° C. until further use.

The epidermal cell (chimney) was left open to ambient laboratory conditions. The dermal cell was filled with receptor solution. Receptor solution for in vitro skin permeations was typically an isotonic saline at physiological pH. The receptor solution may also contain a drug solubilizer, for example, to increase lipophilic drug solubility in the receptor phase. The receptor solution was typically a phosphate buffered saline at approximately pH 7.4 (PBS, pH 7.4; European Pharmacopeia, 3rd Edition, Suppl. 1999, p. 192, No. 4005000) with addition of 2% Volpo N20 (oleyl ether of polyethylene glycol—a nonionic surfactant with HLB 15.5 obtained by ethoxylation (20 moles) of oleyl alcohol (C18:1)). This solubilizer is currently used for in vitro skin permeations and is known not to affect skin permeability (Bronaugh R. L., “Determination of percutaneous absorption by in vitro techniques,” in: Bronaugh R. L., Maibach H. I. (Eds.), “Percutaneous absorption,” Dekker, New York (1985); Brain K. R., Walters K. A., Watkinson A. C., Investigation of skin permeation in vitro, in: Roberts M. S., Walters K. A. (Eds.), Dermal absorption and toxicity assessment, Dekker, New York (1998)).

All cells were mounted in a diffusion apparatus in which the dermal bathing solution (i.e., the receptor solution) was stirred magnetically at approximately 600 RPM and skin surface temperature maintained at 33.0°±1.0° C.

To assure the integrity of each skin section, its permeability to tritiated water was determined before application of the test products. (Franz T. J., et al., “The use of water permeability as a means of validation for skin integrity in in vitro percutaneous absorption studies,” Abst. J Invest Dermatol 94:525 (1990)). Following a brief (0.5-1 hour) equilibrium period, ³H₂O (New England Nuclear, Boston, Mass.; sp. act. ˜0.5 μCi/mL) was layered across (approximately 100-150 μL). After 5 minutes the ³H₂O aqueous layer was removed. At 30 minutes the receptor solution was collected and analyzed for radioactive content by liquid scintillation counting. Skin specimens in which absorption of ³H₂O was less than 1.25 μL-equ (at equilibration) were considered acceptable.

Dosing and Sample Collection.

Franz Cell.

Just prior to dosing with the formulations described herein, the chimney was removed from the Franz Cell to allow full access to the epidermal surface of the skin. The formulations were typically applied to the skin section using a positive displacement pipette set to deliver approximately 6.25 μL (6.25 μL/1 cm²). The dose was spread throughout the surface with the TEFLON® (E. I. Du Pont De Nemours And Company Corporation, Wilmington Del.) tip of the pipette. Five to ten minutes after application the chimney portion of the Franz Cell was replaced. Experiments were performed under non-occlusive conditions. Spare cells were not dosed, but sampled, to evaluate for interfering substances during the analysis.

At pre-selected time intervals after test formulation application (e.g., 2, 4, 8, 12, 24, 32, and 50 hr) the receptor solution was removed in its entirety, replaced with fresh solution (0.1× Phosphate Buffered Saline with Volpo (Croda, Inc., Parsippany, N.J.), and an aliquot taken for analysis. Prior to administration of the topical test formulations to the skin section, the receptor solution was replaced with a fresh solution of Volpo-PBS. (Volpo (Oleth-20) is a non-ionic surfactant known to increase the aqueous solubility of poorly water-soluble compounds. Volpo in the receptor solution ensured diffusion sink conditions during percutaneous absorption, and is known not to affect the barrier properties of the test skin.)

Skin samples from three cadaver skin donors were prepared and mounted onto cells. Typically, each formulation was tested in 4 replicates (3 different donors).

Each formulation was applied, typically, to triplicate sections for each donor. The receptor solution samples were typically collected at 2, 4, 8, 12, 24, 32, and 50 hours after dosing. The receptor solution used was 1:10 PBS+0.1% Volpo. Differences between formulations were evaluated for statistical differences using standard statistical analysis, for example, the Student's t-Test.

After the last sample was collected, the surface was washed twice (0.5 mL volumes) with 50:50 ethanol:water twice to collect un-absorbed formulation from the surface of the skin. Following the wash, the skin was removed from the chamber, split into epidermis and dermis, and each extracted overnight in 50:50 ethanol:water for 24 hours prior to further analysis.

### Automatic Sampling

Automatic sampling was carried out essentially as described under “(a) Franz cell” above, with the exception that multiple cells were used coupled with an automatic sampling system. Skin from a single donor was cut into multiple smaller sections (e.g., punched skin disks cut to approximately 34 mm diameter) large enough to fit on 1.0 cm² Franz diffusion cells (Crown Glass Co., Somerville, N.J.). Skin thickness was typically between 330 and 700 μm, with a mean of 523 μm (+19.5%).

Each dermal chamber was filled to capacity with a receptor solution (e.g., phosphate-buffered isotonic saline (PBS), pH 7.4±0.1, plus 2% Volpo), and the epidermal chamber was left open to ambient laboratory environment. The cells were then placed in a diffusion apparatus in which the dermal receptor solution was stirred magnetically at ˜600 RPM and its temperature maintained to achieve a skin surface temperature of 32.0±1.0° C.

Typically, a single formulation was dosed to 2-3 chambers (comprising the same donor skin) at a target dose of about 5 μL/1.0 cm² using a calibrated positive displacement pipette. At pre-selected times after dosing, (e.g., 2, 4, 8, 12, 24, 32, 48 h) the receptor solution was sampled and a predetermined volume aliquot saved for subsequent analysis. Sampling was performed using a Microette autosampler (Hanson Research, Chatsworth, Calif.).

Following the last receptor solution sample, the surface was washed and the skin collected for analysis as described herein.

Analytical Quantification Methods.

Quantification of ropinirole was by High Performance Liquid Chromatography. (HPLC) with Diode-Array and Mass spectrometry detector (HPLC/MS). Briefly, HPLC was conducted on a HEWLETT-PACKARD® (Hewlett-Packard Company, Palo Alto, Calif.) 1100 Series system with diode-array UV detector with MS detector. A solvent system consisting of 75%: (A) 0.5% Acetic acid, 0.01 M Ammonium Acetate in H₂O and 25% (B) Methanol was run through a C18 Luna column (4.6×100 mm, 3μ, Phenomenex Inc.) at a flow rate of 0.75 mL/min (3.8 minute run duration). Ten micro liters of sample were injected. Peak areas were quantified to concentration using an external standard curve prepared from the neat standard.

(iv) Data Analysis. The permeation studies described herein provide data to obtain different profiles of the transdermal absorption of drugs through the skin as a function of time.

The absolute kinetic profile shows the mean cumulated drug permeated amount (e.g., μg/cm²) as a function of time (e.g., hours) and thus provides an evaluation of the daily absorbed dose (amount of drug transdermally absorbed after 24 hours of permeation). Atenolol and caffeine were used as control substances of high and low permeators.

The relative kinetic profile shows the mean cumulated drug permeated amount (e.g., percent) as a function of time (e.g., hours) and thus allows an evaluation of the percentage of the applied drug that is transdermally absorbed after a given time.

The flux profile shows the mean drug instant flux [e.g., μg/cm²/h] as a function of time (e.g., hours) and provides a time the steady-state flux is reached. This profile also provides an evaluation of the value of this steady-state flux. This value corresponds to the mean flux obtained at steady-state.

These different profiles provide means to evaluate, characterize, and compare formulations, as well as to assess the pharmaceutical efficacy of formulations and consequently, to optimize prototype formulations.

Formulation of Pharmaceutical Compositions.

Experiments performed in support of the present invention showed that the order of addition of the components was not significant, that is, the components may be added in essentially any order during manufacturing processes. Further, nitrogen sparging is not required during manufacturing of the pharmaceutical compositions of the present invention but use of nitrogen sparging is also not counter-indicated. In the pharmaceutical formulations described herein below, the solubility of the active ingredient (e.g., ropinirole or ropinirole hydrochloride) was not an issue.

Following here is an exemplary description of the manufacturing process used to make the pharmaceutical compositions of the present invention. Generally, the organic solution was prepared, comprising, for example, solvent/cosolvent (e.g., ethanol/water/propylene glycol), penetration enhancer, preservative/antioxidant, and thickening (or gelling) agent. The organic solution was mixed (e.g., using mechanical mixing) to yield a homogeneous, clear solution. The active agent, ropinirole, was then added to the solution and the solution mixed to obtain a homogeneous, clear active organic solution. Water was then added quantum sufficiat (q.s.). If desired, the pH was then adjusted to a specified pH. In some cases, water was added and pH was adjusted before the addition of ropinirole so that ropinirole was not exposed to high local pH variations; although timing of the pH adjustment was not an issue. Some compositions were purged of air by nitrogen bubbling before ropinirole was dissolved; however, as noted above, such nitrogen sparging was not required. As noted above, the components may be added in essentially any order during manufacturing processes.

One exemplary method of manufacturing is as follows. Ethanol, propylene glycol, diethylene glycol monoethylether and myristyl alcohol were weighed and added successively. The organic solution was mixed using mechanical mixing (e.g., magnetic stirring). The resulting organic solution was clear and homogeneous. Ropinirole HCl was added to the organic solution and mixed until solution was achieved. The resulting solution was clear and homogeneous. Then 85-90% of the total amount of water was added to the active organic solution and mixed. The resulting solution was clear and homogeneous. Triethanolamine (typically about 20% w/w aqueous solution) was added and the solution mixed until the solution was homogeneous. The resulting solution was clear and homogeneous with a pH, for example, of between 7.85 and 8.0. When the pH was within the desired specification range, water was added q.s. to the solution to obtain final appropriate weight percents of components and the pH of the final solution measured. If the pH was below the desired pH (e.g., pH 7.85), further triethanolamine solution was added and the pH of the final solution remeasured. Typically, total triethanolamine amount did not exceed 5.50% w/w.

### EXAMPLE 1

### Intrinsic in vitro Permeation Results

Table 1 describes formulations that were evaluated for in vitro permeation. Evaluation of in vitro permeation was carried out as described in the Materials and Methods section using Franz cells.

TABLE 1

| | | | Drug |
|---|---|---|---|
| | | | Concentration |
| Formulation | Drug | Formulation (%) | (%) |
| A | Ropinirole | EtOH(45)/Water(40)/PG(10) | 5 |
| | HCl | | |
| B | Ropinirole | EtOH(45)/Water(40)/PG(10) | 5 |
| | Base | | |

In Table 1, ethanol is EtOH and polyethylene glycol is PG. The formulation and drug concentration percentages are given in weight percent. Two comparable formulations were made for each of two control substances, caffeine and atenolol, at a drug concentration of 1% for each drug in each formulation. For Formulation B, the ropinirole free base was generated in situ from ropinirole HCl by adjusting the pH of Formulation B to pH 9.5-10.0 using NaOH. The primary purpose of using these formulations was to evaluate intrinsic permeation and to compare the free base and salt forms of ropinirole.

Human cadaver skin was used for the permeation studies using Franz cells as described in the Materials and Methods.

The flux results of the permeation analysis using the formulations in Table 1 are presented FIG. 1. In FIG. 1, the vertical axis is Flux (μg/cm²/hr), the horizontal axis corresponds to sampling times (in hours), flux values for ropinirole HCl are represented using a square, flux values for ropinirole free base are represented using a circle, flux values for caffeine are represented using an upright triangle, and flux values for Atenolol are represented using an inverted triangle. Mass balance recovery data from the permeation analysis is presented in FIG. 2. In FIG. 2, the vertical axis is the percent dose recovered and the horizontal axis shows the amounts of recovered dose in the receptor chamber fluid, the dermis, the epidermis, the surface wash, and total recovery (respectively, groups left to right in FIG. 2). The four vertical bars in each group correspond, respectively, to ropinirole HCl, ropinirole free base, caffeine, and atenolol.

The data presented in FIGS. 1 and 2 demonstrate that the ropinirole hydrochloride salt did not permeate well in its native substantially protonated form (in these solutions) and the ropinirole free base demonstrated good permeation characteristics (in these solutions).

In addition, these data demonstrate, for the ropinirole free base formulations presented in Table 1, that the ropinirole has a peak flux of 3.5 μg/cm²/hr showing that delivery of 4.8 mg of ropinirole can be achieved using solutions formulations in 24 hours when applied to a skin area of 57 cm². Approximately 20% of the ropinirole remained in the epidermis after 48 hours. Bioavailability of ropinirole was about 40% in the receptor chamber fluid. These results suggested that the gel formulation provides a sustained depot of ropinirole when used, for example, in a once daily application of gel to subject skin surface.

These in vitro permeation results for the free base of ropinirole demonstrated adequate flux in an un-optimized formulation for use in pharmaceutical transdermal delivery of the drug. In this initial study, the ropinirole hydrochloride salt did not demonstrate skin permeation characteristics in its native form; however, formulation modifications described herein below result in good permeation characteristics for the ropinirole hydrochloride salt.

These results demonstrate that ropinirole in a gel provided sufficient transdermal flux for transdermal gel compositions to be used for therapeutic delivery of ropinirole.

### EXAMPLE 2

### Ropinirole Skin Permeation pH Sensitivity

Table 2 presents exemplary components of ropinirole gel formulations used in the following experiments.

TABLE 2
Composition of Formulations (% w/w)

| | | Formu- | | Formu- |
|---|---|---|---|---|
| General | | lation | Formulation | lation |
| Component | Specific Component | A1 | B1 | C1 |
| Solvent | Absolute Ethanol | 45.00 | 45.00 | 45.00 |
| | Purified Water | 23.79 | 21.84 | 14.08 |
| Cosolvent | Propylene glycol | 20.00 | 20.00 | 20.00 |
| Penetration | Diethylene glycol | 5.00 | 5.00 | 5.00 |
| enhancer | monoethylether | | | |
| | Myristyl alcohol | 1.00 | 1.00 | 1.00 |
| Gelling agent | Hydroxypropyl | 1.50 | 1.50 | 1.50 |
| | cellulose (Klucel HF) | | | |
| pH Modifier | Triethanolamine | 0.29 | 2.24 | — |
| | 20% w/w | | | |
| | 50% w/w | — | — | 10.00 |
| Active Drug | Ropinirole HCl* | 3.42 | 3.42 | 3.42 |

| | | | |
|---|---|---|---|
| Final pH | ~6.0 | 7.12 | 7.90 |
| Total | 100.00 | 100.00 | 100.00 |

*Ropinirole HCl 3.42% (MW = 296.84) corresponds to Ropinirole free base 3% Ropinirole HCL 3.42% (MW = 296.84) corresponds to Ropinirole free base 3% (MW = 260.38), ratio 1.14.

### Formulations A1, B1, and C1 were made essentially as described above in the Materials and Methods.

Transdermal delivery of ropinirole using Formulations A1, B1, and C1 was evaluated using an apparatus for automated sampling (described in the Materials and Methods Section). Individual gel amounts applied to tested skin samples were approximately 10 mg. Studies were performed according to OECD (Organization for Economic Cooperation and Development) guidelines (Organization for Economic Co-operation and Development (OECD), Environment Directorate. “Guidance document for the conduct of skin absorption studies,” OECD series on testing and assessment, No. 28. Paris, version 05 March 2004). The results presented in Table 3 show the mean values of cumulative delivered amount of ropinirole after 24 hours. The total amount of ropinirole in each of Formulations A1, B1, and C1 was the same.

TABLE 3
Ropinirole Cumulative Delivery After 24 hours Permeation

| | N | | |
|---|---|---|---|
| | (number of | Time | Mean Cumulative |
| Formulation | samples) | (in hours) | Delivery (μg/cm² ± SD) |
| A1 | 4 | 24 | 3.45 ± 2.39 |
| B1 | 4 | 24 | 7.33 ± 5.31 |
| C1 | 4 | 24 | 63.03 ± 20.04 |

Further, the absolute kinetic delivery profile of ropinirole over the 24 hour permeation are presented in FIG. 3. In FIG. 3, the vertical axis is Cumulated Drug Permeated (μg/cm²), the horizontal axis is Time (in hours), the data points for Formulation A1 are presented as diamonds, the data points for Formulation B1 are presented as squares, the data points for Formulation C1 are presented as upright triangles, and error bars (SD, standard deviation) are presented for each data point.

The data presented in Table 3 and FIG. 3 illustrate the surprising discovery that transdermal permeation of ropinirole HCl is sensitive to the pH of the formulation in which it is contained. The experimental findings presented in Example 1 demonstrated low transdermal permeation of ropinirole HCl compared to ropinirole free base in formulations where the pH was not adjusted. The data presented in Example 1, FIGS. 1 and 2, illustrated greater transdermal permeation of ropinirole free base relative to ropinirole HCl in those formulations. In contrast, the data in the present example demonstrated the efficient transdermal permeation of ropinirole HCl at about pH 8. The effect of increasing pH from pH 6.0, to pH 7.0, to pH 8.0 can be seen in FIG. 3 to correspond to increasing transdermal permeation of ropinirole HCl.

Data from this study demonstrate that transdermal ropinirole delivery is pH-sensitive. Bioavailability was doubled (from 2% to 4%) when the pH of the formulation was increased from pH˜6 to pH˜7 (Formulation A versus Formulation B). A huge increase was observed between pH˜7 and pH˜8 (Formulation B versus Formulation C) because the transdermal bioavailability was multiplied by 9: from 4% to 36% (significant, p=0.002). Overall, a pH difference of two units resulted in an almost 20-fold increase of the transdermal bioavailability: from 2% to 36% (p=0.001).

The pH of human skin is typically about pH 4.5-6.0. One advantage of obtaining transdermal permeation of ropinirole at pH values closer to the physiological pH of human skin than the pKa of free base ropinirole is a possible reduction in skin irritation potential at the site of application of transdermal formulations comprising ropinirole. Further, as can be seen from the above data, a large increase in bioavailability of the ropinirole was seen in formulations having pH values of between about pH 7 and about pH 8.

### EXAMPLE 3

### Ionization Profiles for Ropinirole

The effect of pH on transdermal delivery of ropinirole was assessed. The permeation profile was compared to the ionization profile, which was obtained from experimental titration.

Experiments performed in support of the present invention have shown that increasing pH of a 3.4% ropinirole HCl formulation from 6 to 8 resulted in increase in drug delivery by almost 20-fold. However, the pKa of ropinirole is 9.7. Therefore, such a jump in drug delivery was unexpected, because, for example, as depicted on FIG. 4A, the theoretical difference in ropinirole ionization between 6 and 8 (FIG. 4A, squares, Theoretical Ionization Profile) is small compared to ropinirole delivery (FIG. 4A, diamonds, Ropinirole Delivery).

The ionization curve and pKa appeared to be applicable to completely aqueous solutions. However, many of the ropinirole formulations of the present invention contain only about 15-20% water. The remaining preponderant solvents are typically a short chained alcohol (e.g., ethanol) and a cosolvent (e.g., propylene glycol). In those solvents, measured pH was apparent, and appeared shifted compared to theoretical pH.

The following formulation was used for titration to determine the experimental ionization profile of ropinirole in a hydroalcoholic base: Ropinirole hydrochloride* 3.42% w/w, myristyl alcohol 1.00% w/w, diethylene glycol monoethylether 5.00% w/w, propylene glycol 20.00% w/w, absolute ethanol 45.00% w/w, and purified water 25.58% w/w (total 100; * ropinirole HCl 3.42% (MW=296.84) corresponds to ropinirole free base 3% (MW=260.38), ratio 1.14). The formulation was not gellified.

The ropinirole HC1 solution was titrated with NaOH 0.1 M solution. The solvent was the same as the formulation, in order to keep a constant composition. NaOH was chosen so as to limit dilution of the titrated formulation; but no dilution correction was made. The formulation was titrated by 0.5-1 mL increments where the pH change was small. Increments were reduced to 0.1 mL near the equivalence point. The pH was monitored with a glass electrode (Mettler Toledo InLab 432, Mettler-Toledo, Inc., Columbus, Ohio), and recorded with a Mettler Toledo MP. 230 pH meter (Mettler-Toledo, Inc., Columbus, Ohio).

Based on the titration curve, the ionization rate [BH] was calculated according to Henderson-Hasselbalch equation for weak base:

$$[ {BH}^{+} ] = \frac{{10}^{pKa - pH}}{1 + {10}^{pKa - pH}}$$

The experimental ionization profile for ropinirole (shown in FIG. 4B) provided a pKa=8.0, where pH=pKa when [BH+]=50%.

This information, taken in conjunction with the data presented in Example 2 suggested that the alcohol/water solvent causes an apparent shift in the pKa of ropinirole. This apparent pKa shift illustrates an advantage of the pharmaceutical gel formulations described herein for transdermal use because the gel formulations of the present invention can be adjusted to pH values closer to the physiological pH of human skin (wherein the mean value typically lies in the range pH 5.4-5.9), thus reducing the possibility of skin irritation caused by the gel formulations of the present invention, and still deliver pharmaceutically efficacious amounts to a subject via transdermal permeation. Further observations and advantages related to the pKa shift of ropinirole in non-aqueous media are discussed in Example 6 herein below.

### EXAMPLE 4

### Drug Concentration Effects

Table 4 presents exemplary components of ropinirole gel formulations used in the following experiments.

TABLE 4
Composition of Formulations (% w/w)

| | | Formu- | | Formu- |
|---|---|---|---|---|
| General | | lation | Formulation | lation |
| Component | Specific Component | A2 | B2 | C2 |
| Solvent | Absolute Ethanol | 45.00 | 45.00 | 45.00 |
| | Purified Water | 14.08 | 22.99 | 20.34 |
| Cosolvent | Propylene glycol | 20.00 | 20.00 | 20.00 |
| Penetration | Diethylene glycol | 5.00 | 5.00 | 5.00 |
| enhancer | monoethylether | | | |
| | Myristyl alcohol | 1.00 | 1.00 | 1.00 |
| Gelling agent | Hydroxypropyl | 1.50 | 1.50 | 1.50 |
| | cellulose (Klucel HF) | | | |
| pH Modifier | Triethanolamine | 10.00 | 2.80 | — |
| | 50% w/w | | | |
| | 1M HCl | — | — | 4.16 |
| Active Drug | Ropinirole HCl* | 3.42 | 1.71 | — |
| | Ropinirole Free Base | — | — | 3.00 |

| | | | |
|---|---|---|---|
| Final pH | 7.90 | 7.86 | 7.71 |
| Total | 100.00 | 100.00 | 100.00 |

Formulations A2, B2, and C2 were made essentially as described above in the Materials and Methods.

The concentration of 3.4% of ropinirole HCl is equivalent to a concentration of approximately 3% ropinirole free base.

Transdermal delivery of ropinirole using Formulations A2, B2, and C2 was evaluated using an apparatus for automated sampling (described in the Materials and Methods Section). Individual gel amounts applied to tested skin samples were approximately 10 mg. Studies were performed according to OECD (Organization for Economic Cooperation and Development) guidelines (Organization for Economic Co-operation and Development (OECD), Environment Directorate. “Guidance document for the conduct of skin absorption studies,” OECD series on testing and assessment, No. 28. Paris, version 05 March 2004). The results presented in Table 5 show the mean values of cumulative delivered amount of ropinirole after 24 hours.

TABLE 5
Ropinirole Cumulative Delivery After 24 hours Permeation

| | N | | Mean Cumulative |
|---|---|---|---|
| | (number of | Time | Delivery |
| Formulation | samples) | (in hours) | (μg/cm² ± SD) |
| A2 | 4 | 24 | 28.35 ± 5.50 |
| B2 | 4 | 24 | 21.86 ± 9.65 |
| C2 | 4 | 24 | 17.93 ± 8.01 |

Further, the absolute kinetic delivery profile of ropinirole delivery over the 24 hour permeation are presented in FIG. 5. In FIG. 5, the vertical axis is Cumulated Drug Permeated (μg/cm²), the horizontal axis is Time (in hours), the data points for Formulation A2 are presented as diamonds, the data points for Formulation B2 are presented as squares, the data points for Formulation C2 are presented as upright triangles, and error bars (SD, standard deviation) are presented for each data point.

The data presented in Table 5 and FIG. 5 illustrate the surprising discovery that transdermal permeation of ropinirole HCl is sensitive to the concentration of the ropinirole HCl in the formulation, when the formulations are at the same pH (e.g., pH 7.8). A strict dose/response curve would predict that the formulation of ropinirole HCl at half the ropinirole concentration (i.e., 1.7%) would have half of the cumulative transdermal permeation ropinirole compared to the formulation of ropinirole HCl at the unit dose (i.e., 3%). However, this was not the case. In this example, the cumulative transdermal permeation of ropinirole with the lower concentration formulation of ropinirole HCl (i.e., 1.7%) was approximately 75% of the transdermal permeation of ropinirole with the higher concentration formulation of ropinirole HCl (i.e., 3.4%).

One possible explanation for this effect may be that it is a salt effect or a counter ion effect on skin permeability of ropinirole, for example, NaCl may be present as a neutralization byproduct and may have a positive impact on permeability of ropinirole.

One advantage of obtaining a higher percentage transdermal permeation of ropinirole HCl at pH values closer to the apparent pKa of ropinirole in an alcohol/water solvent (i.e., apparent pKa 7.7) is the ability to make pharmaceutically efficacious gel formulations using lower concentrations of ropinirole while maintaining the ability to achieve the necessary steady state concentration of ropinirole in the blood of a subject being treated with such gel formulations.

### EXAMPLE 5

### Antioxidant Effects on Ropinirole Skin Permeation

The effect of an antioxidant in ropinirole gel formulations was evaluated. Table 6 presents specific, exemplary formulations used in the following experiments.

TABLE 6
Composition of Formulations (% w/w)

| | | Formu- | | Formu- |
|---|---|---|---|---|
| General | | lation | Formulation | lation |
| Component | Specific Component | A3 | B3 | C3 |
| Solvent | Absolute Ethanol | 45.00 | 45.00 | 45.00 |
| | Purified Water | 22.99 | 20.85 | 20.76 |
| Cosolvent | Propylene glycol | 20.00 | 20.00 | 20.00 |
| Penetration | Diethylene glycol | 5.00 | 5.00 | 5.00 |
| enhancer | monoethylether | | | |
| | Myristyl alcohol | 1.00 | 1.00 | 1.00 |
| Gelling agent | Hydroxypropyl | 1.50 | 1.50 | 1.50 |
| | cellulose (Klucel HF) | | | |
| pH Modifier | Triethanolamine | 2.80 | 4.54 | — |
| | (50% w/w) | | | |
| | 1M HCl | — | — | 2.44 |
| | 0.1M HCl | — | — | 2.40 |
| Antioxidant | Sodium Metabisulfite | — | 0.40 | 0.40 |
| Active Drug | Ropinirole HCl* | 1.71 | 1.71 | — |
| | Ropinirole Free Base | — | — | 1.50 |

| | | | |
|---|---|---|---|
| Final pH | 7.86 | 8.10 | 8.00 |
| Total | 100.00 | 100.00 | 100.00 |

*Ropinirole HCl 1.71% (MW = 296.84) corresponds to Ropinirole free base 1.5% (MW = 260.38), ratio 1.14.

Formulations A3, B3, and C3 were made essentially as described above in the Materials and Methods.

The concentration of 1.7% of ropinirole HCl is equivalent to a concentration of approximately 1.5% ropinirole free base.

Transdermal delivery of ropinirole using Formulations A3, B3, and C3 was evaluated using an apparatus for automated sampling (described in the Materials and Methods Section). Individual gel amounts applied to tested skin samples were approximately 10 mg. Studies were performed according to OECD (Organization for Economic Cooperation and Development) guidelines (Organization for Economic Co-operation and Development (OECD), Environment Directorate. “Guidance document for the conduct of skin absorption studies,” OECD series on testing and assessment, No. 28. Paris, version 05 March 2004). The results presented in Table 7 show the mean values of cumulative delivered amount of ropinirole after 24 hours.

TABLE 7
Ropinirole Cumulative Delivery After 24 hours Permeation

| | N | | Mean Cumulative |
|---|---|---|---|
| | (number of | Time | Delivery |
| Formulation | samples) | (in hours) | (μg/cm² ± SD) |
| A3 | 4 | 24 | 30.78 ± 9.77 |
| B3 | 3 | 24 | 39.20 ± 2.89 |
| C3 | 3 | 24 | 26.27 ± 2.23 |

Further, the absolute kinetic delivery profile of ropinirole over the 24 hour permeation are presented in FIG. 6. In FIG. 6, the vertical axis is Cumulated Drug Permeated (μg/cm²), the horizontal axis is Time (in hours), the data points for Formulation A3 are presented as diamonds, the data points for Formulation B3 are presented as squares, the data points for Formulation C3 are presented as upright triangles, and error bars (SD, standard deviation) are presented for each data point.

The data presented in Table 7 and FIG. 6 illustrate that addition of the antioxidant sodium metabisulfite (NaMET) does not impair ropinirole transdermal bioavailability. The data illustrate the surprising discovery that NaMET appears to improve transdermal bioavailability by about 25%.

The results of ropinirole steady-state flux after 24 hours of permeation are presented in Table 8. The steady-state flux was reached for all formulations. Steady-state flux was calculated by linear regression of the time points 14-19-24 h in FIG. 7.

TABLE 8
Ropinirole Steady-State Flux After 24 hours Permeation

| | N | | Mean Cumulative |
|---|---|---|---|
| | (number of | Time | Delivery |
| Formulation | samples) | (in hours) | (μg/cm²h ± SD) |
| A3 | 4 | 14-24 | 0.92 ± 0.09 |
| B3 | 3 | 14-24 | 1.11 ± 0.22 |
| C3 | 3 | 14-24 | 0.98 ± 0.22 |

The results of ropinirole instant flux over 24 hour permeation are presented in FIG. 7. In FIG. 7, the vertical axis is Drug Instantaneous Flux (μg/cm²/hour), the horizontal axis is Time (in hours), the data points for Formulation A3 are presented as diamonds, the data points for Formulation B3 are presented as squares, the data points for Formulation C3 are presented as upright triangles, and error bars (SD, standard deviation) are presented for each data point. Accordingly, FIG. 7 presents data for flux rate over time.

Drug instantaneous flux was measured by determining the difference between the concentration at a first time point (e.g., 14 hours) and the subsequent time point (e.g., 19 hours) and thus is a measure of how much ropinirole permeated the skin since the previous time point.

The data presented in FIG. 7 supports the surprising discovery that addition of sodium metabisulfite (NaMET) improves transdermal flux of ropinirole. As seen in FIG. 7, 0.4% NaMET (Formulation B3) does not impair ropinirole transdermal bioavailability, compared to absence of antioxidant (Formulation A3). On the contrary, the addition of 0.4% NaMET appears to improve transdermal bioavailability of ropinirole by about 25%. Further, these results demonstrate that the ropinirole HCl salt (Formulation B3) performs 50% better (p=0.002) than the ropinirole free base (Formulation C3) in these formulations.

Experiments performed in support of the present invention demonstrated a similar effect on bioavailability due to the addition of 0.4% NaMET in comparable formulations to those set forth in Table 6 but which comprised 3.42% ropinirole HCl and 3.00% ropinirole free base.

One advantage of obtaining a higher percentage transdermal permeation of ropinirole HCl in the presence of the antioxidant sodium metabisulfite is the ability to enhance bioavailability via transdermal permeation of ropinirole.

### EXAMPLE 6

### Further Investigation of the Effect of pH on Ropinirole Transdermal Delivery.

The effect of pH on transdermal delivery of ropinirole was further evaluated. Table 9 presents exemplary formulations used in the following experiments.

TABLE 9
Composition of Formulations (% w/w)

| | | Formu- | | Formu- |
|---|---|---|---|---|
| General | | lation | Formulation | lation |
| Component | Specific Component | A4 | B4 | C4 |
| Solvent | Absolute Ethanol | 45.00 | 45.00 | 45.00 |
| | Purified Water | 20.58 | 17.04 | 13.68 |
| Cosolvent | Propylene glycol | 20.00 | 20.00 | 20.00 |
| Penetration | Diethylene glycol | 5.00 | 5.00 | 5.00 |
| enhancer | monoethylether | | | |
| | Myristyl alcohol | 1.00 | 1.00 | 1.00 |
| Gelling agent | Hydroxypropyl | 1.50 | 1.50 | 1.50 |
| | cellulose (Klucel HF) | | | |
| pH Modifier | 1M Sodium | 3.10 | 6.64 | 10.00 |
| | hydroxide | | | |
| Antioxidant | Sodium Metabisulfite | 0.40 | 0.40 | 0.40 |
| Active Drug | Ropinirole HCl* | 3.42 | 3.42 | 3.42 |

| | | | |
|---|---|---|---|
| Final pH | 7.37 | 7.96 | 8.57 |
| Total | 100.00 | 100.00 | 100.00 |

*Ropinirole HCl 3.42% (MW = 296.84) corresponds to Ropinirole free base 3% (MW = 260.38), ratio 1.14.

Formulations A4, B4, and C4 were made essentially as described above in the Materials and Methods.

Transdermal delivery of ropinirole using Formulations A4, B4, and C4 was evaluated using an apparatus for automated sampling (described in the Materials and Methods Section). Individual gel amounts applied to tested skin samples were approximately 11 mg for Formulation A4 and approximately 10 mg for each of Formulations B4 and C4. Studies were performed according to OECD (Organization for Economic Cooperation and Development) guidelines (Organization for Economic Co-operation and Development (OECD), Environment Directorate. “Guidance document for the conduct of skin absorption studies,” OECD series on testing and assessment, No. 28. Paris, version 05 March 2004). The results presented in Table 10 show the mean values of cumulative delivered amount of ropinirole after 24 hours.

TABLE 10
Ropinirole Cumulative Delivery After 24 hours Permeation

| | N | | Mean Cumulative |
|---|---|---|---|
| | (number of | Time | Delivery |
| Formulation | samples) | (in hours) | (μg/cm² ± SD) |
| A4 | 4 | 24 | 7.33 ± 1.96 |
| B4 | 4 | 24 | 11.12 ± 1.78 |
| C4 | 4 | 24 | 17.52 ± 5.96 |

Further, the relative kinetic delivery profile of ropinirole delivery over the 24 hour permeation, which illustrates ropinirole bioavailability, are presented in FIG. 8. In FIG. 8, the vertical axis is Cumulated Drug Permeated (%), the horizontal axis is Time (in hours), the data points for Formulation A4 are presented as diamonds, the data points for Formulation B4 are presented as squares, the data points for Formulation C4 are presented as upright triangles, and error bars (SD, standard deviation) are presented for each data point.

The data presented in Table 10 and FIG. 8 illustrate that the pH of the formulation had a clear effect on ropinirole bioavailability, for example, pH increase from approximately pH 7.5 to 8.0 results in 50% increase of drug delivery (significant, p=0.03), and further increase to approximately pH 8.5 results in additional 60% increase in drug delivery (not significant, p=0.09).

In other words, linear pH increase results in almost linear drug delivery increase in the range of approximately pH 7 to approximately pH 8, as shown in Examples 2 and 3 herein above. This is consistent with the apparent ionization profile of ropinirole (see, for example, Example 3 herein above, where the pKa of ropinirole in non-aqueous media shifted from 9.7 to about 7.7), where the decrease in ionization corresponds to the increase in drug delivery (see, FIG. 9). In FIG. 9, the left vertical axis is Cumulative ropinirole delivery (μg/cm²), the horizontal axis is pH, and the right vertical axis is Ropinirole Ionization Rate (%); ropinirole delivery data points are presented as diamonds and the apparent ropinirole ionization profile data points are presented as circles.

In this example, all formulations pH were adjusted with NaOH and not triethanolamine (TEA). Some impact was seen on the bioavailability of ropinirole when NaOH was used. The reference formulation at pH 8 with NaOH displayed about 6.4% bioavailability, compared to 20% bioavailability when the formulation was pH adjusted with TEA.

These data demonstrate the sensitivity of transdermal permeation of ropinirole to the pH of the formulation. The data support that a preferred range of final formulation pH for the transdermal delivery of ropinirole is about pH 7 to about pH 9, with a more preferred range of final formulation pH of between about pH 7.5 to about pH 8.5.

### EXAMPLE 7

### Effects of Buffering Agent Concentration on the Transdermal Delivery of Ropinirole

The effect of the concentration of the buffering agent (pH modifier) on the transdermal delivery of ropinirole was evaluated. Table 11 presents exemplary formulations used in the following experiments.

TABLE 11
Composition of Formulations (% w/w)

| | | Formu- | | Formu- |
|---|---|---|---|---|
| General | | lation | Formulation | lation |
| Component | Specific Component | A5 | B5 | C5 |
| Solvent | Absolute Ethanol | 45.00 | 45.00 | 45.00 |
| | Purified Water | 19.68 | 18.68 | 17.28 |
| Cosolvent | Propylene glycol | 20.00 | 20.00 | 20.00 |
| Penetration | Diethylene glycol | 5.00 | 5.00 | 5.00 |
| enhancer | monoethylether | | | |
| | Myristyl alcohol | 1.00 | 1.00 | 1.00 |
| Gelling agent | Hydroxypropyl | 1.50 | 1.50 | 1.50 |
| | cellulose (Klucel HF) | | | |
| pH Modifier | Triethanolamine | 4.00 | 5.00 | 6.40 |
| | (50% w/w) | | | |
| Antioxidant | Sodium Metabisulfite | 0.40 | 0.40 | 0.40 |
| Active Drug | Ropinirole HCl* | 3.42 | 3.42 | 3.42 |

| | | | |
|---|---|---|---|
| Final pH | 7.86 | 7.93 | 8.06 |
| Total | 100.00 | 100.00 | 100.00 |

*Ropinirole HCl 3.42% (MW = 296.84) corresponds to Ropinirole free base 3.00% (MW = 260.38), ratio 1.14.

Formulations A5, B5, and C5 were made essentially as described above in the Materials and Methods.

Transdermal delivery of ropinirole using Formulations A5, B5, and C5 was evaluated using an apparatus for automated sampling (described in the Materials and Methods Section). Individual gel amounts applied to tested skin samples were approximately 10 mg. Studies were performed according to OECD (Organization for Economic Cooperation and Development) guidelines (Organization for Economic Co-operation and Development (OECD), Environment Directorate. “Guidance document for the conduct of skin absorption studies,” OECD series on testing and assessment, No. 28. Paris, version 05 March 2004). The results presented in Table 12 show the mean values of cumulative delivered amount of ropinirole after 24 hours.

TABLE 12
Ropinirole Cumulative Delivery After 24 hours Permeation

| | N | | |
|---|---|---|---|
| | (number of | Time | Mean Cumulative |
| Formulation | samples) | (in hours) | Delivery (μg/cm² ± SD) |
| A5 | 4 | 24 | 60.22 ± 15.46 |
| B5 | 4 | 24 | 57.56 ± 9.76 |
| C5 | 4 | 24 | 49.92 ± 12.27 |

Further, the absolute kinetic delivery profile of ropinirole delivery over the 24 hour permeation are presented in FIG. 10. In FIG. 10, the vertical axis is Cumulated Drug Permeated (μg/cm²), the horizontal axis is Time (in hours), the data points for Formulation A5 are presented as diamonds, the data points for Formulation B5 are presented as squares, the data points for Formulation C5 are presented as upright triangles, and error bars (SD, standard deviation) are presented for each data point.

The results of ropinirole steady-state flux after 24 hours of permeation are presented in Table 13. The steady-state flux was reached for all formulations. Steady-state flux was calculated by linear regression of the time points 14-19-24 h in FIG. 11.

TABLE 13
Ropinirole Steady-State Flux After 24 hours Permeation

| | N | | Mean Cumulative |
|---|---|---|---|
| | (number of | Time | Delivery |
| Formulation | samples) | (in hours) | (μg/cm²h ± SD) |
| A5 | 4 | 14-24 | 1.68 ± 0.26 |
| B5 | 4 | 14-24 | 1.59 ± 0.14 |
| C5 | 4 | 14-24 | 1.67 ± 0.22 |

The results of ropinirole instantaneous flux over 24 hour permeation are presented in FIG. 11. In FIG. 11, the vertical axis is Drug Instant Flux (μg/cm²/hour), the horizontal axis is Time (in hours), the data points for Formulation A5 are presented as diamonds, the data points for Formulation B5 are presented as squares, the data points for Formulation C5 are presented as upright triangles, and error bars (SD, standard deviation) are presented for each data point. Accordingly, FIG. 11 presents data for flux rate over time.

The data presented in this example illustrated that differences in TEA concentration in the tested range (4-6.4%) did not result in significant differences in at approximately pH 8 for the formulations. Permeation data confirm that drug delivery and transdermal bioavailability were not statistically different between Formulations A5, B5, and C5. However, transdermal bioavailability of these formulations ranged between about 29% and about 33%, which was about four times the transdermal bioavailability of the formulations whose pH was adjusted with NaOH alone (see above). These results suggest a beneficial effect of TEA, and similar buffering agents, as compared to use of NaOH alone.

### EXAMPLE 8

### General Formulation Guidelines For Preferred Transdermal Gel Compositions

Based on experiments performed in support of the present invention, the following general formulation guidelines were determined for transdermal gel compositions comprising ropinirole for pharmaceutical applications. Percentages given in Table 14 are approximate percentages. Variations on the compositions will be clear to one of ordinary skill in the art in view of the teachings of the present specification. Adjustment to volume to obtain total weight percent typically employs addition of alcohol, water, and/or cosolvent q.s.

TABLE 14
Composition of Formulations (% w/w)

| | | More | |
|---|---|---|---|
| General | | Preferred | |
| Component | Preferred Range | Range | Exemplary Component |
| Solvent: | | | |
| Alcohol | 30%-70% | 40%-60% | Absolute Ethanol |
| Water | 10%-60% | 15%-40% | Purified Water |
| Cosolvent | 10%-60% | 15%-40% | Propylene glycol |
| Penetration | 0.1%-10% | 1.0%-7% | Diethylene glycol |
| enhancer | | | monoethylether and |
| | | | Myristyl alcohol (5:1) |
| Antioxidant | 0.01%-5% | 0.1%-0.5% | Sodium metabisulfite |
| Gelling Agent | 0.5%-5% | 1%-3% | Hydroxypropyl |
| | | | cellulose |
| pH Modifier | 1%-10% | 3%-5% | Triethanolamine (50% |
| | | | w/w aqueous solution) |
| Active Drug | 0.5%-5% | 1%-3.5% | Ropinirole (free base |
| | | | equivalents*) |
| Final pH | 7-9 | 7.5-8.5 | |

*Ropinirole HCl 1.71% (MW = 296.84) corresponds to Ropinirole free base 1.5% (MW = 260.38), ratio 1.14.

The primary vehicle of the transdermal gel formulations of the present invention was a gellified hydroalcoholic mixture (e.g., ethanol/water gellified with hydroxypropyl cellulose). The transdermal gel formulations of the present invention contained a pharmaceutically effective amount of active drug (e.g., ropinirole), typically had a final pH of between about 7.0 and 8.5, and, in some embodiments, further comprised permeation enhancer(s) and/or antioxidant(s). In Table 14 the exemplary ranges are given as weight percents, with the exception of the final pH, wherein the range is presented as a target pH range.

The solvent is typically a mixture of solvents, for example, alcohol and water, with possible additional cosolvent(s), for example, propylene glycol. The vapor pressure of the solvent is typically such that the majority of the solvent is capable of evaporating at body temperature. The normal range of human body temperature is typically about 31-34° C., with an average of about 32° C. The gelling agent is typically present in an amount to impart a three-dimensional, cross-linked matrix to the solvent. The pH of the formulation is adjusted, for example, by addition of aqueous triethanolamine before the final volume of the formulation is brought to 100 g (basis for weight percent). Alternately or in addition, pH can be adjusted by titration and final total weight adjusted q.s., for example, with purified water.

Accordingly, in one embodiment of the present invention includes a formulation of ropinirole in a hydroalcoholic gel, pH about 7.5 to about 8.5, which may further comprise antioxidant(s) and penetration enhancer(s).

### EXAMPLE 9

### Stability of Ropinirole Compositions

The following experiment visually investigated the effect of antioxidants and chelating agents on coloration of ropinirole hydrochloride formulations. Experiments performed in support of the present invention demonstrated that ropinirole compositions change color in a range of light yellow to dark violet/black. It has also been demonstrated that coloration is linked to ropinirole degradation. Accordingly, stability of ropinirole formulations can be assessed using coloration as surrogate marker to assess stability of ropinirole formulations.

Formulations containing 3.42% wt ropinirole hydrochloride (corresponding to 3.00% wt ropinirole free base) were tested. The formulations were similar to Formulation A2 (described in Table 4, herein above) with the addition of the following agents: Edetic acid (EDTA); Butylhydroxytoluene (BHT); Propyl gallate (ProGL); Sodium metabisulfite (NaMET); and combinations thereof. Edetic acid and edetates are chelating agents that are commonly considered as antioxidant synergists. BHT, ProGL and NaMET are considered as true antioxidants. The concentration of each agent was typically about 0.10% (w/w). A blank formulation (i.e., containing no antioxidants) was used for comparison. The test formulations were as shown in Table 15.

TABLE 15
Stability Test Formulations

| | | | | |
|---|---|---|---|---|
| | EDTA | BHT | ProGL | NaMET |
| Sample | 0.10% wt | 0.10% wt | 0.10% wt | 0.10% wt |

| | | | | |
|---|---|---|---|---|
| 1 | X | | | |
| 2 | | X | | |
| 3 | | | X | |
| 4 | | | | X |
| 5 | X | X | | |
| 6 | X | | X | |
| 7 | X | | | X |
| 8 | | X | X | |
| 9 | | X | | X |
| 10 | | | X | X |

Aliquots of the formulations were placed in sealed transparent glass-vials for ten days at 60° C. This unusual high-temperature condition was selected to further enhance discrimination of formulations. Solutions were checked for visual aspect and color.

The following ranking of the samples, in terms of best stability, was observed (starting with the best stability): 7>10>(4 & 9)>(1 & 5 & 6)>2>(3 & 8). The results of the analysis showed excellent ropinirole stability for all formulations containing NaMET (i.e., 4, 7, 9, and 10). When NaMET was used in combination with other agents, there was some increase in stability of ropinirole based on visual inspection. Some synergistic effects were observed with NaMET in combination with ProGL, BHT, and EDTA.

These results indicate that the formulations of the present invention provide stable, pharmaceutically acceptable formulations of ropinirole.

Further stability tests may be performed, for example, as follows. Aliquots of the formulations are placed in isolation at room temperature, under accelerated conditions (˜40° C.), and under refrigerated conditions. The formulations are tested for overall stability and/or stability of individual components (e.g., on days 0, 7, 14, 21, 28, 90, 180 (±1 day)). Each formulation is tested in triplicate on each evaluation day.

In addition, aliquots may be tested in a variety of container means, for example, foil packages, laminated collapsible tubes, vials, and/or metered dose delivery devices.

### EXAMPLE 10

### Skin Irritation Studies

The degree of skin irritation caused by the ropinirole formulations of the present invention are first tested in standard animal models. For example, skin irritation studies are carried out in rabbits using a modified Draize irritation protocol (see, e.g., Balls, M, et al., “The EC/HO international validation study on alternatives to the Draize eye irritation test,” Toxicology In Vitro 9:871-929 (1995); Draize J, et al., “Methods for the study of irritation and toxicity of substances applied topically to the skin and mucous membranes,” J Pharmacol Exp Ther 82:377-390 (1944); and CEC, Collaborative Study on the Evaluation of Alternative Methods to the Eye Irritation Test. Doc. I/632/91/V/E/1/131/91 Part I and II (2001)).

Formulations to be tested include, for example, different formulations of ropinirole free base (at one or more concentrations), ropinirole HCl (at one or more concentrations), or combinations thereof wherein the above identified components of the formulations of the present invention (e.g., different ratios of alcohol/water, variations on the alcohol used in the alcohol/water solvent, different types and concentration of cosolvent(s), different types and concentrations of permeation enhancer(s), different types and concentrations of antioxidant(s), different types and concentrations of thickener(s)) and/or conditions (e.g., pH, and compositions stored for different periods of time) are varied. Mineral oil is typically used as a negative control.

The mean primary irritation score for each treatment is calculated according to the selected protocol.

Preliminary indications (for example, the pH effects, Example 2, and dosage effects, Example 4, described above) suggest that the irritation encountered upon transdermal administration of ropinirole using the formulations of the present invention is minimal.

### EXAMPLE 11

### In vivo Human Transdermal Permeation Studies

The efficacy of transdermal delivery for therapeutic applications using the ropinirole gel formulations of the present invention are evaluated using standard clinical procedures. For example, healthy, human participants are selected typically representing a variety of ages, races, and gender. Ropinirole gel formulations are provided for daily application by the participants to skin surface. Blood concentration of ropinirole is determined by blood draw at pre-selected time intervals (e.g., hourly, multiple daily, daily). Determination of ropinirole concentration in plasma is determined by standard procedures (e.g., “Liquid chromatographic determination of 4-(2-di-N,N-propylaminoethyl)-2-(3H)-indolone in rat, dog, and human plasma with ultraviolet detection,” Swagzdis, J. E., et al., Journal of Pharmaceutical Sciences, Volume 75(1), pages 90-91 (1986)). The ability to deliver steady state, therapeutic concentrations of ropinirole using the formulations of the present invention is determined by plotting blood concentration of ropinirole against elapsed time over a pre-selected time period (e.g., days or weeks).

Alternately, or in addition, urine concentrations of ropinirole or related metabolites may also be determined (“Application of thermospray liquid chromatography-mass spectrometry and liquid chromatography-tandem mass spectrometry for the identification of cynomolgus monkey and human metabolites of SK&F 101468, a dopamine D2 receptor agonist,” Beattie, I. G., et al., Journal of Chromatography (1989), Volume 474(1), pages 123-138 (1988)).

Alternately, or in addition, human participants are evaluated for therapeutic effects of ropinirole on, for example, Parkinson's Disease, as well as for side effects of the method of delivery (e.g., skin irritation) and known side effects typically associated with oral administration of ropinirole (e.g., involuntary movements, dizziness, drowsiness, excessive tiredness, headache, upset stomach, heartburn, vomiting, constipation, frequent urination, dry mouth, decreased sexual ability, hallucinations, fainting, high temperature, rigid muscles, confusion, increased sweating, irregular heartbeat, chest pain, swelling of the feet, ankles, or lower legs, cold or flu-like symptoms, changes in vision, and/or falling asleep while eating, having a conversation, or in the middle of another activity). Such a clinical trial may include, for example, comparison to treatment by standard oral delivery of ropinirole (see, e.g., “Dosing with ropinirole in a clinical setting,” Korczyn, A. D., et al., Acta Neurol. Scand. Volume 106, pages 200-204 (2002)).

### EXAMPLE 12

### Transdermal Ropinirole Pharmacokinetics

A Phase 1 clinical trial was conducted using a 1.5% free base equivalent gel to determine the pharmacokinetics of ropinirole delivered via the transdermal routes as described in Example 11. This Phase 1 study was a single-center, open-label study. The study consisted of one day of oral dosing of IR ropinirole followed by a washout period and then randomization. Subjects were randomized with equal chance to receive one of three regimens of daily application of ropinirole transdermal gel for 5 days. The gel formulation contained 1.71% ropinirole hydrochloride (1.5% ropinirole expressed as free base equivalents) in a hydroalcoholic gel matrix. The study was conducted in 30 subjects. Following screening and baseline procedures, eligible subjects were entered into the study. Treatment A was followed by a minimum of a 4-day wash out period, and then, 5 days of a once daily application of either Treatment B, C or D.

Treatment A: Immediate release ropinirole dosed orally as 0.25 mg three times every six hours for one day.

Treatment B: 55 μL ropinirole transdermal gel containing 0.75 mg ropinirole applied over 3×3 cm area on the shoulder or abdomen.

Treatment C: 220 μL ropinirole transdermal gel containing 3.0 mg ropinirole applied over 6×6 cm area on the shoulder or abdomen.

Treatment D: 220 μL ropinirole hydrochloride transdermal gel containing 3.0 mg ropinirole applied over 8.5×8.5 cm area on the shoulder or abdomen.

Blood samples were collected predose and at specified time points up to 72 hours following the oral IR ropinirole dose, and pre-dose and during 24 hours post-dose on the first and fifth day of application of ropinirole transdermal gel (Day 8 and Day 12), pre-dose before the second, third and fourth application of the gel treatments (Day 9, 10 and 11), and through 96 hours post the last dose (Days 13 to 16) for the determination of plasma ropinirole concentrations.

The mean concentration-time profiles of plasma ropinirole following different treatments are plotted in FIGS. 14 and 15.

As can be seen from the predicted data in FIGS. 13 and the experimental data in FIG. 15, dosage forms of the present invention provide for the delivery of ropinirole over a prolonged period of time, for example, such that once-a-day administration of the drug is possible. Further, the reduced ratio of Cₘₐₓ to Cₘᵢₙ (at steady state), as well as the slower oscillation between Cₘₐₓ and Cₘᵢₙ (at steady state) provided by the dosage forms of the present invention may provide more consistent plasma concentration for subjects treated with a dosage form of the present invention versus multi-time per day dosing (e.g., three times a day) using an oral dosage form.

### EXAMPLE 13

### Dermal Irritation and Sensation Studies

The local irritation of the current clinical formulation of transdermal ropinirole HCl was evaluated using the modified Draize scale as described in Example 10. Data indicated that the local tolerability of this formulation is acceptable and support the use of the formulation in humans. Ropinirole HCl gel at up to 5% was mildly irritating with once daily application for 14 days to Hanford mini-pigs. Additionally ropinirole HCl was categorized as a mild sensitizer based on guinea pigs induced (with and without Freund's Complete Adjuvant) and challenged with 5% ropinirole HCl.

As is apparent to one of skill in the art, various modification and variations of the above embodiments can be made without departing from the spirit and scope of this invention. Such modifications and variations are within the scope of this invention.

We claim:

1. A gel for pharmaceutical drug delivery, comprising:
a therapeutically effective amount of ropinirole, or a pharmaceutically acceptable salt thereof;
a primary vehicle comprising a mixture of water and at least one short-chain alcohol;
at least one antioxidant; and
at least one buffering agent, wherein (i) the pH of the gel is between about pH 7 and about pH 8.5, and (ii) the gel is adapted for application to the surface of skin.

2. The gel of claim 1, wherein the ropinirole is free base ropinirole.

3. The gel of claim 1, wherein the ropinirole is a pharmaceutically acceptable salt of ropinirole.

4. The gel of claim 3, wherein the pharmaceutically acceptable salt is ropinirole HCl.

5. The gel of claim 1, wherein the ropinirole is presented at a concentration of about 0.5 to about 10 weight percent of ropinirole free base equivalents.

6. The gel of claim 5, wherein the ropinirole is presented at a concentration of about 1 to about 5 weight percent of ropinirole free base equivalents.

7. The gel of claim 1, wherein the short-chain alcohol is selected from the group consisting of ethanol, propanol, isopropanol, and mixtures thereof.

8. The gel of claim 7, wherein the short-chain alcohol is ethanol.

9. The gel of claim 8, wherein the ethanol is present at a concentration of about 30 to about 70 weight percent and the water is present at a concentration of about 10 to about 60 weight percent.

10. The gel of claim 9, wherein the ethanol is present at a concentration of about 40 to about 60 weight percent and the water is present at a concentration of about 10 to about 40 weight percent.

11. The gel of claim 1, wherein the primary vehicle further comprises a non-volatile solvent.

12. The gel of claim 1 1, wherein the non-volatile solvent is a glycol or glycerin.

13. The gel of claim 12, wherein the glycol is propylene glycol.

14. The gel of claim 13, wherein the concentration of propylene glycol is about 10 to about 60 weight percent.

15. The gel of claim 14, wherein the concentration of propylene glycol is about 15 to about 40 weight percent.

16. The gel of claim 1, wherein the primary vehicle further comprises a gelling agent.

17. The gel of claim 16, wherein the gelling agent is selected from the group consisting of modified cellulose and gums.

18. The gel of claim 17, wherein the modified cellulose is selected from the group consisting of hydroxypropyl cellulose, hydroxyethyl cellulose, and carboxymethyl cellulose.

19. The gel of claim 18, wherein the modified cellulose is hydroxypropyl cellulose.

20. The gel of claim 19, wherein the concentration of hydroxypropyl cellulose is between about 0.5 and about 3 weight percent.

21. The gel of claim 20, wherein the concentration of hydroxypropyl cellulose is between about 1 and about 2 weight percent.

22. The gel of claim 1, wherein the primary vehicle further comprises a penetration enhancer.

23. The gel of claim 22, wherein the penetration enhancer is present at a concentration of about 0.1 to about 10 weight percent.

24. The gel of claim 23, wherein the penetration enhancer is present at a concentration of about 1 to about 7 weight percent.

25. The gel of claim 23 wherein the penetration enhancer is a mixture of diethylene glycol monoethylether and myristyl alcohol in, respectively, a 5:1 ratio weight/weight.

26. The gel of claim 1, wherein the antioxidant is present at a concentration of about 0.01 to about 1 weight percent.

27. The gel of claim 26, wherein the antioxidant is present at a concentration of about 0.1 to about 0.5 weight percent.

28. The gel of claim 26, wherein the antioxidant comprises sodium metabisulfite.

29. The gel of claim 1, wherein the buffering agent is present at a concentration of about 1 to about 10 weight percent.

30. The gel of claim 29, wherein the buffering agent is present at a concentration of about 3 to about 5 weight percent.

31. The gel of claim 29, wherein the buffering agent comprises triethanolamine.

32. The gel of claim 1, wherein
the therapeutically effective amount of ropinirole, or a pharmaceutically acceptable salt thereof, is between about 0.5 to about 10 weight percent of ropinirole free base equivalents;
the primary vehicle comprises between about 10 to about 60 weight percent of water, between about 30 to about 70 weight percent ethanol, between about 10 and about 60 weight percent propylene glycol, and between about 0.1 and about 10 weight percent of a 5:1 (weight to weight) mixture of diethylene glycol monoethylether and myristyl alcohol, wherein the primary vehicle is gellified with between about 0.5 and about 3 weight percent of hydroxypropyl cellulose;
the antioxidant comprises between about 0.01 and about 1 weight percent of sodium metabisulfite; and
the buffering agent comprises triethanolamine between about 1 to about 10 weight percent, wherein the pH of the gel is between about pH 7 and about pH 8.5.

33. A unit dose container, comprising inner and outer surfaces, wherein the gel for pharmaceutical drug delivery of claim 1 is contained by the inner surface of the container.

34. The unit dose container of claim 33, wherein the container is a packet or a vial.

35. The unit dose container of claim 34, wherein the inner surface of the container further comprises a liner.

36. The unit dose container of claim 35, wherein the packet is a flexible, foil packet and the liner is a polyethylene liner.

37. A multiple dose container, comprising inner and outer surfaces, wherein the gel for pharmaceutical drug delivery of claim 1 is contained by the inner surface of the container.

38. The multiple dose container of claim 37, wherein the multiple dose container dispenses fixed or variable metered doses.

39. The multiple dose container of claim 37, wherein the multiple dose container is a stored-energy metered dose pump or a manual metered dose pump.

40. A composition for pharmaceutical drug delivery, comprising
a therapeutically effective amount of ropinirole, or a pharmaceutically acceptable salt thereof, in a hydroalcoholic vehicle comprising water, a short chain alcohol, and at least one buffering agent, wherein (i) the pH of the composition is between about pH 7 and about pH 8.5, (ii) transdermal flux of the ropinirole, in the hydroalcoholic vehicle, across skin is greater than the transdermal flux of an equal concentration of ropinirole in an aqueous solution of essentially equivalent pH over an essentially equivalent time period, and (iii) the skin is the flux rate controlling membrane.

41. The composition of claim 40, wherein the hydroalcoholic vehicle further comprises an antioxidant.

42. The composition of claim 40, wherein the hydroalcoholic vehicle is gellified.

43. A composition for pharmaceutical drug delivery, comprising
a therapeutically effective amount of ropinirole, or a pharmaceutically acceptable salt thereof, in a hydroalcoholic vehicle comprising water, and a short chain alcohol, wherein (i) the ropinirole has an apparent pKa of about 8.0 or less compared to a theoretical pKa of ropinirole in water of about pKa 9.7, and (ii) the composition is formulated for application to the surface of skin.

44. The composition of claim 43, wherein the ropinirole is a pharmaceutically acceptable salt thereof.

45. The composition of claim 44, wherein the ropinirole is ropinirole HCl.

46. The composition of claim 43, further comprising an antioxidant.

47. The composition of claim 43, wherein the hydroalcoholic vehicle further comprises one or more component selected from the group consisting of a cosolvent, a penetration enhancer, a buffering agent and a gelling agent.

48. A method of manufacturing a gel for pharmaceutical drug delivery, comprising
mixing the following components to yield a homogeneous gel, wherein the pH of the gel is between about pH 7 and about pH 8.5:
a therapeutically effective amount of ropinirole, or a pharmaceutically acceptable salt thereof;
a primary vehicle comprising water, at least one short-chain alcohol, and at least one gelling agent;
at least one antioxidant; and
at least one buffering agent;
to provide a gel suitable for pharmaceutical delivery of ropinirole.

49. The method of claim 48, wherein the primary vehicle further comprises at least one cosolvent and/or at least one penetration enhancer.

50. The method of claim 48, the method further comprising dispensing the gel into one or more containers.

51. The method of claim 50, wherein the container is a unit dose container.

52. The method of claim 51, wherein the container is a flexible, foil packet, further comprising a liner.

53. The method of claim 50, wherein the container is a multiple dose container.

54. A method for administering an active agent to a human subject in need thereof, the method comprising:
providing a gel for pharmaceutical drug delivery, comprising:
a therapeutically effective amount of ropinirole, or a pharmaceutically acceptable salt thereof;
a primary vehicle comprising a gellified mixture of water and at least one short-chain, alcohol;
at least one antioxidant; and
at least one buffering agent, wherein the pH of the gel is between about pH 7 and about pH 8.5;
applying one or more daily dose of the gel to a skin surface of the subject in an amount sufficient for the ropinirole to achieve therapeutic concentration in the bloodstream of the subject.

55. The method of claim 54, wherein the human subject is in need of ropinirole therapy to treat a movement disorder.

56. The method of claim 55, wherein the human subject is in need of ropinirole therapy to treat a condition selected from the group consisting of Parkinson's Disease, Restless Legs Syndrome, Tourette's Syndrome, Chronic Tic Disorder, Essential Tremor, and Attention Deficit Hyperactivity Disorder.

57. The method of claim 54, wherein the gel has an amount of ropinirole free base equivalents between about 3 and about 5 weight percent and up to about 1 gram of the gel is applied daily to a skin surface area of between about 50 to about 1000 cm².

58. The method of claim 54, wherein the gel has an amount of ropinirole free base equivalents of about 1.5 weight percent, wherein up to about 1.5 grams of the gel is applied daily to a skin surface area of between about 70 to about 300 cm².

59. The method of claim 54, wherein the gel has an amount of ropinirole free base equivalents of about 3 weight percent, wherein up to about 0.25 grams of gel is applied daily to a skin surface area between about 50 and 300 cm².

60. The method of claim 51, wherein the gel dose is applied in a single or in divided doses.

61. A dosage form for delivery of ropinirole to a subject comprising,
a dose of ropinirole, wherein said dosage form is configured to provide (i) steady-state delivery of ropinirole with once-a-day dosing, and (ii) a steady-state ratio of Cₘₐₓ/Cₘᵢₙ that is less than about 1.75 when the subject's plasma level concentration of ropinirole is at steady-state (C_{SS}).

62. The dosage form of claim 61, wherein Cₘₐₓ/Cₘᵢₙ is less than about 1.5.

63. The dosage form of claim 61, wherein Cₘₐₓ/Cₘᵢₙ is less than about 1.3.

64. The dosage form of claim 61, wherein said once-a-day dosing is performed for about 2 successive days or more.

65. The dosage form of claim 61, wherein said dosage form comprises a dose of ropinirole between about 0.5 to about 10 weight percent of ropinirole free base equivalents, and said dosage form is a pharmaceutical composition for non-occlusive, transdermal drug delivery.

66. A dosage form of claim 61, for use in preparation of a medicament for treatment of a movement disorder.

67. A dosage form for delivery of ropinirole to a subject comprising,
a dose of ropinirole, wherein said dosage form is configured to provide (i) steady-state delivery of ropinirole with once-a-day dosing, and (ii) a steady-state oscillation of Cₘₐₓ to Cₘᵢₙ of greater than about 8 hours when the subject's plasma level concentration of ropinirole is at steady-state (C_{SS}).

68. The dosage form of claim 67, wherein the steady-state oscillation of Cₘₐₓ to Cₘᵢₙ of greater than about 10 hours.

69. The dosage form of claim 67, wherein the steady-state oscillation of Cₘₐₓ to Cₘᵢₙ of greater than about 12 hours.

70. The dosage form of claim 67, wherein said once-a-day dosing is performed for about 2 successive days or more.

71. The dosage form of claim 67, wherein said dosage form comprises a dose of ropinirole between about 0.5 to about 10 weight percent of ropinirole free base equivalents, and said dosage form is a pharmaceutical composition for non-occlusive, transdermal drug delivery.

72. A dosage form according to claim 67, for use in preparation of a medicament for treatment of a movement disorder.
